(19) United States
(12) Patent Application Publication
(10) Pub. No.: US 2020/0155436 A1
(43) Pub. Date: May 21, 2020

(54) COMPOSITION CONTAINING QUATERNARY AMMONIUM COMPOUND, ESPECIALLY FOR PRODUCING CARE AND CLEANING FORMULATIONS

(71) Applicant: Evonik Operations GmbH, Essen (DE)

(72) Inventors: Christian Hartung, Essen (DE); Matthias Mentel, Dortmund (DE); Verena Dahl, Bergisch Gladbach (DE); Peter Schwab, Essen (DE); Kristin Klostermann, Essen (DE); Patrick Winter, Mülheim an der Ruhr (DE); Jochen Kleinen, Heinsberg (DE)

(73) Assignee: Evonik Operations GmbH, Essen (DE)

(21) Appl. No.: 16/636,678

(22) PCT Filed: Aug. 14, 2018
(86) PCT No.: PCT/EP2018/071980

§ 371 Date: Feb. 5, 2020

(30) Foreign Application Priority Data
Sep. 6, 2017 (EP) 17189528.7

(51) Int. Cl. A61K 8/41; A61K 8/06; A61K 8/34; A61K 8/37; A61Q 5/02; A61Q 5/12; A61Q 5/00; A61Q 19/10

(52) U.S. Cl. CPC A61K 8/416; A61K 8/068; A61K 8/345; A61Q 19/10; A61Q 5/02; A61Q 5/12; A61Q 5/006; A61K 8/37

(57) ABSTRACT

The invention relates to a composition comprising:
A) at least one non-silicone-containing quaternary ammonium compound,
B) at least one cosmetic oil,
C) at least one nonionic surfactant,
D) at least one nonaqueous solvent,
E) water.

### CROSS REFERENCE TO RELATED APPLICATIONS

This application is a 35 U.S.C. § 371 U.S. national phase entry of International Application No. PCT/EP2018/071980 having an international filing date of Aug. 14, 2018, which claims the benefit of European Application No. 17189528.7 filed Sep. 6, 2017, each of which is incorporated herein by reference in its entirety.

### FIELD

The invention relates to a composition comprising:

A) at least one non-silicone-containing quaternary ammonium compound,
B) at least one cosmetic oil,
C) at least one nonionic surfactant,
D) at least one nonaqueous solvent,
E) water,
and also the use of such compositions, for example in a process for producing formulations.

### BACKGROUND

WO 2008155075 describes a cosmetic preparation comprising (a) at least one surfactant selected from non-alkoxylated anionic, zwitterionic or amphoteric surfactants, (b) a microemulsion and (c) at least one cationic polymer.

EP 1715833 describes a microemulsion having a mean particle diameter of 5 to 250 nm, comprising (a) 5 to 50 wt % of at least one particular alkyl and/or alkenyl oligoglycoside carboxylic acid salt, (b) 5 to 50 wt % of an oil component and (c) 0 to 15 wt % of mono- and/or polyfunctional alcohols having 1 to 4 carbon atoms, where the sum of components (a) and (b) makes up 10 to 55 wt % of the total composition.

WO 2008155073 describes a cosmetic preparation comprising (a) at least one surfactant selected from anionic, zwitterionic or amphoteric surfactants, (b) a microemulsion and (c) at least one cationic polymer.

DE 19755488 describes microemulsions comprising (a) 5 to 30 wt % of oil bodies, (b) 5 to 80 wt % of anionic and/or nonionic emulsifiers and (c) 12 to 30 wt % of polyols, with the proviso that the stated amounts together with water add up to 100 wt %.

The disadvantages of the compositions described in the prior art are that the cosmetic formulations thereof only have a small conditioning effect on skin and hair, or that they only have a comparatively low affinity for particular surfaces. In addition, the compositions of the prior art commonly very strongly lower the viscosity in surfactant formulations such as shampoos, and thickening of the formulation is extremely difficult.

### SUMMARY

It was an object of the invention to overcome at least one of the disadvantages from the prior art.

### DETAILED DESCRIPTION

It has been found that, surprisingly, the compositions described below are able to solve the problem addressed by the invention.

The present invention provides compositions comprising at least one non-silicone-containing quaternary ammonium compound, at least one cosmetic oil, at least one nonionic surfactant, at least one nonaqueous solvent and water.

The invention further provides for the use of the compositions according to the invention for the production of formulations and also the formulations comprising the microemulsions according to the invention.

An advantage of the present invention is that otherwise water-insoluble oils can be converted into a clear water-dilutable form.

A further advantage of the present invention is that oils can be incorporated into cosmetic formulations, which oils cannot be incorporated into cosmetic formulations in their pure form or diluted in solvents due to the lack of compatibility.

Yet another advantage of the present invention is that relatively high proportions of water-insoluble oils can be incorporated into cosmetic formulations clearly and in a storage-stable manner.

A further advantage of the present invention is that the microemulsions, because of their low viscosity, are easy to process and to incorporate into formulations. Simple stirring-in at low temperatures is sufficient, for example at 25° C.

A further advantage of the present invention is that the water-insoluble oils achieve better performance in formulations. The microemulsions of the present invention enhance the conditioning properties of the oils present, such as combability, softness, volume, shapeability, handleability, detanglability of undamaged and damaged hair, and shine effect.

Yet another advantage of the present invention is that the formulations have a particularly good conditioning effect in cosmetic, dermatological and pharmaceutical formulations. Yet another advantage of the present invention is that the formulations are able to produce a pleasant skin sensation in cosmetic formulations.

A further advantage of the present invention is that the formulations described here can be formulated from polyether-free constituents, and cosmetic formulations can be provided which are substantially free from alkoxylated constituents.

A further advantage of the products according to the invention is that they are silicone-free, yet in this respect have a very similar sensory profile on the hair or the skin.

A further advantage of the present invention is that the formulations described here can improve the shine of the hair.

Yet another advantage of the present invention is that the thickenability of the described formulations is good.

A further advantage of the present invention is that further very hydrophobic oils such as vegetable oils, which can be chosen freely, can be incorporated in a certain proportion to give a clear formulation and hence can likewise be formulated easily.

A further advantage of the present invention is that rapid drying of hair can be achieved in the case of use in cosmetic formulations.

Yet another advantage of the present invention is that the compositions according to the invention do not need any preservatives.

All conditions such as pressure and temperature, for example, unless stated otherwise, are standard conditions (20° C., 1 bar).

In connection with the present invention, the term “fatty acids” shall especially be understood to mean formic acid, acetic acid, propionic acid, butyric acid, valeric acid, caproic acid, enanthic acid, caprylic acid, pelargonic acid, capric acid, lauric acid, myristic acid, pentadecanoic acid, palmitic acid, margaric acid, stearic acid, nonadecanoic acid, arachic acid, behenic acid, lignoceric acid, cerotic acid, montanic acid, melissic acid, undecylenoic acid, myristoleic acid, palmitoleic acid, petroselic acid, oleic acid, elaidic acid, vaccenic acid, gadoleic acid, eicosenoic acid, cetoleic acid, erucic acid, nervonic acid, linoleic acid, alpha-linolenic acid, gamma-linolenic acid, calendulic acid, punicic acid, alpha-eleostearic acid, beta-eleostearic acid, arachidonic acid, timnodonic acid, clupanodonic acid, cervonic acid, vernolic acid, ricinoleic acid, particular preference being given to those having a chain length of 6 to 24, preferably 6 to 22 and especially 8 to 18 carbon atoms; the same applies to the carbon base skeleton for the term “fatty alcohols” used in connection with the invention.

Unless stated otherwise, all percentages (%) given are percentages by mass.

The present invention provides a composition comprising:

A) at least one non-silicone-containing quaternary ammonium compound,
B) at least one cosmetic oil,
C) at least one nonionic surfactant,
D) at least one nonaqueous solvent,
E) water.

A preferred composition of the present invention is characterized in that it is a microemulsion.

Microemulsions are thermodynamically stable mixtures of water (aqueous phase), oil (water-immiscible phase) and surfactant (solubilizer).

Microemulsions preferred in accordance with the invention have a domain size of the disperse phase of less than 1000 nm, especially less than 500 nm, the determination of domain size being conducted with the aid of scattering methods known to those skilled in the art, as described, for example, in P. Lindner and Th. Zemb, “Neutrons, X-Rays and Light: Scattering Methods Applied to Soft Condensed Matter”, Elsevier Science & Technology, November 2002, or O. Glatter and O. Kratky, “Small-angle X-ray Scattering” Academic Press Inc, December 1982.

For cosmetic or topical applications in particular, preference is given to compositions which are substantially free from alkoxylated compounds. The term “substantially free from alkoxylated compounds” in connection with the present invention is to be understood to mean that the composition has no notable amounts of alkoxylated compounds which exert a surface-active effect. This is especially understood to mean that alkoxylated compounds are present in amounts of less than 1 wt %, preferably less than 0.1 wt %, more preferably less than 0.01 wt %, based on the total composition, in particular there are no detectable amounts.

A preferred composition of the present invention is characterized in that it contains

A) in an amount of 1 wt % to 40 wt %, preferably in an amount of 1.5 wt % to 30 wt %, more preferably in an amount of 5 wt % to 25 wt %,
B) in an amount of 5 wt % to 60 wt %, preferably in an amount of 10 wt % to 50 wt %, more preferably in an amount of 15 wt % to 40 wt %,
C) in an amount of 5 wt % to 50 wt %, preferably in an amount of 10 wt % to 40 wt %, more preferably in an amount of 15 wt % to 35 wt %,
D) in an amount of 2 wt % to 50 wt %, preferably in an amount of 3 wt % to 40 wt %, more preferably in an amount of 5 wt % to 30 wt %, and
E) in an amount of 1 wt % to 60 wt %, preferably in an amount of 3 wt % to 50 wt %, more preferably in an amount of 5 wt % to 45 wt %,
where the percentages by weight refer to the total composition and in particular with the proviso that the mass of the component B) is greater than the mass of the component A) in the composition.

As component A), for example, quaternary ammonium compounds having at least one linear and/or branched, saturated or unsaturated alkyl chain can be used, for instance alkyltrimethylammonium halides such as, e.g., cetyltrimethylammonium chloride or bromide or behenyltrimethyl ammonium chloride, or else dialkyldimethylammonium halides such as, e.g., distearyldimethylammonium chloride. Furthermore as component A), monoalklyamido quats such as, e.g., palmitamidopropyltrimethylammonium chloride, or corresponding dialkylamido quats, can be used. Furthermore as component A), quaternary ester compounds can be used, which may be quaternized fatty acid esters based on mono-, di- or triethanolamine. Component A) may also be alkylguanidinium salts or imidazolinium salts.

A preferred composition of the present invention is characterized in that

A) is selected from the group of the ester quats and imidazolinium salts, preferably ester quats, especially liquid ester quats and liquid imidazolinium salts, liquid ester quats being especially preferred.

In the context of the present invention, the term “ester quat” is understood to mean a chemical compound containing both a quaternary nitrogen atom and an ester bond in the cationic portion of an ion pair. This is preferably understood to mean a class of surface-active quaternary ammonium compounds having the general formula R¹¹R¹²R¹³R¹⁴N⁺X⁻, characterized in that at least one of the radicals R¹¹ to R¹⁴, which is characterized in that it has more than 4 carbon atoms, is bonded to the charged group via ester bonds C(O)O— or OC(O)—, and X⁻ is understood to mean any anionic counterion.

R¹¹ is a saturated or unsaturated, straight-chain, branched or cyclic, optionally substituted hydrocarbon radical that is optionally interrupted by oxygen atoms, nitrogen atoms or carboxyl groups
R¹² is a saturated or unsaturated, straight-chain, branched or cyclic, optionally substituted hydrocarbon radical that is optionally interrupted by oxygen atoms, nitrogen atoms or carboxyl groups
R¹³ is a saturated or unsaturated, straight-chain, branched or cyclic, optionally substituted hydrocarbon radical that is optionally interrupted by oxygen atoms, nitrogen atoms or carboxyl groups
R¹⁴ is a saturated or unsaturated, straight-chain, branched or cyclic, optionally substituted hydrocarbon radical that is optionally interrupted by oxygen atoms, nitrogen atoms or carboxyl groups.

In connection with the present invention, the term “liquid ester quats” is understood to mean ester quats having, at 1 bar, a melting point of 40° C. or lower, more preferably 25° C. or lower and very preferably 10° C. or lower. If the ester quats present in the composition are mixtures of ester quats, the melting point relates to the melting point of the mixture of all ester quats present in the formulation. The same applies to imidazolinium salts.

A preferred composition of the present invention is characterized in that

A) is selected from the group of the liquid ester quats consisting of quaternized fatty acid alkanolamine ester salts, more preferably from the groups of the quaternized fatty acid ethanolamine ester salts and the quaternized fatty acid isopropanolamine ester salts, very preferably from the group of the quaternized fatty acid isopropanolamine ester salts based on dimethylmono-, methyldi- or triisopropanolamine.

This has the advantageous effect of improving the storage stability of the composition according to the invention over time.

An especially preferred composition of the present invention is characterized in that

A) is selected from the group of the liquid ester quats comprising compounds of the general formula (I)

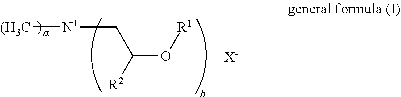

where R¹ is an acyl radical of an at least monounsaturated fatty acid having a chain length of 18 to 24 carbon atoms or the acyl radical of isostearic acid or ricinoleic acid,
where R² is an alkyl radical having 1 to 6 carbon atoms, preferably methyl, ethyl, propyl or isopropyl, more preferably methyl, and
where X⁻ is a counterion to the positive charge on the quaternary nitrogen group, where doubly or triply negatively charged ions are also to be included here,
where a=1 to 3 and b=1 to 3, preferably a=1.7 to 2.3 and b=1.7 to 2.3,
with the proviso that a+b=4.

The inventive composition is outwardly neutral in terms of its electrical charge, since the charges on the ester quats are neutralized by corresponding counterions X—. Suitable according to the invention are all counterions that are able to compensate for the charge on the quats. In the context of the present invention, X— is preferably selected from the group of the cosmetically acceptable anions, especially the group of chloride, sulfate, phosphate, methylsulfate, ethylsulfate, methanesulfonate, ethanesulfonate, tosylate, acetate, lactate and citrate.

If b is >1, the R¹ radicals may be the same or different.

R¹ as acyl radical of an at least monounsaturated fatty acid with a chain length from 18 to 24 carbon atoms can contain one or more, for example two or three, double bonds. Compositions preferred according to the invention are characterized in that R¹ as acyl radical of an at least monounsaturated fatty acid with a chain length of 18 to 24 carbon atoms is selected from the acyl radicals of the acids from the group comprising oleic acid, elaidic acid, vaccenic acid, gadoleic acid, icosenoic acid, cetoleic acid, erucic acid, nervonic acid, linolic acid, alpha-linolenic acid, gamma-linolenic acid, calendulic acid, punicic acid, alpha-elaeostearic acid, beta-elaeostearic acid, arachidonic acid, timnodonic acid, clupanodonic acid and cervonic acid, wherein oleic acid is particularly preferred.

It is also possible in accordance with the invention to use mixtures of these carboxylic acids.

Preferred compositions according to the invention comprise at least one compound of the general formula (I) with a=1.7 to 2.3 and b=1.7 to 2.3, more preferably a=b=2.

A composition particularly preferred in accordance with the invention is characterized in that R¹ is the acyl radical of oleic acid and a=1.7 to 2.3 and b=1.7 to 2.3, more preferably a=b=2.

Liquid imidazolinium salts preferably contained in the composition according to the invention are 1-alkylamidoimidazolinium and 1-alkoxyalkylimidazolinium salts of the general formulae (II) and (III)

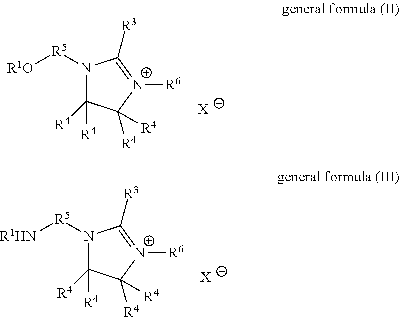

where
R³ is an optionally branched, optionally unsaturated alkyl radical having 1 to 30 carbon atoms optionally interrupted by oxygen atoms,
R⁴ are each independently hydrogen or alkyl, preferably butyl, propyl, ethyl, methyl or hydrogen,
R⁵ is a divalent, saturated or unsaturated, straight-chain, branched or cyclic, optionally substituted hydrocarbon radical optionally interrupted by oxygen atoms, nitrogen atoms or carboxyl groups, preferably ethylene,
R⁶ is hydrogen or an optionally branched, optionally unsaturated alkyl radical optionally containing oxygen atoms or nitrogen atoms and having 1 to 30 carbon atoms, preferably having 1 to 12 carbon atoms, more preferably having 1 to 4 carbon atoms, especially preferably ethyl or methyl,
and
X⁻ is a counterion to the positive charge on the quaternary nitrogen group, where doubly or triply negatively charged ions are also to be included here.

Component B) is a cosmetic oil.

In connection with the present invention, the term “cosmetic oil” is understood to mean water-immiscible liquids suitable for production of cosmetic formulations. In the context of the present invention, water-immiscible signifies that, at room temperature, aqueous mixtures of cosmetic oils at oil concentrations of 0.5-99.5 vol %, based on the total mixture, result in cloudiness visible to the human eye or in the formation of two or more phases. In the context of the present invention, furthermore, cosmetic oils are preferably characterized in that they have an interfacial tension with respect to water of >5 mN/m. Cosmetic oils can be for example oleochemistry-based or silicone chemistry-based.

Preferably according to the invention, the composition according to the invention contains cosmetic oils selected from the group of fatty alcohols, esters of linear fatty acids with linear or branched fatty alcohols, esters of branched fatty acids with linear or branched fatty alcohols, esters of linear fatty acids with unbranched or branched polyhydric alcohols, esters of branched fatty acids with unbranched or branched polyhydric alcohols, esters of linear fatty acids with unbranched or branched alcohols, esters of branched fatty acids with unbranched or branched alcohols, esters of alkylhydroxycarboxylic acids with linear or branched fatty alcohols. In addition, mono-, di- or triglycerides in liquid or solid form. In addition, esters of carboxylic acids, aromatic carboxylic acids or dicarboxylic acids with linear or branched fatty alcohols, unbranched or branched polyhydric alcohols or unbranched or branched alcohols. In addition, linear, cyclic or branched hydrocarbons, with or without substituents, with or without double bonds. In addition, vegetable oils, carbonates with unbranched or branched alcohols, carbonates with unbranched or branched polyhydric alcohols, carbonates with linear or branched fatty alcohols. In addition, ethers with or without alkoxy groups, or silicone oils with or without organic modification. In addition, mixtures of these oils in any ratios. Preferably, esters of linear fatty acids with linear or branched fatty alcohols, esters of branched fatty acids with linear or branched fatty alcohols, esters of linear fatty acids with unbranched or branched polyhydric alcohols, esters of branched fatty acids with unbranched or branched polyhydric alcohols, esters of linear fatty acids with unbranched or branched alcohols, esters of branched fatty acids with unbranched or branched alcohols. In addition, mono-, di- or triglycerides in liquid or solid form. In addition, esters of carboxylic acids, aromatic carboxylic acids or dicarboxylic acids with linear or branched fatty alcohols, unbranched or branched polyhydric alcohols or unbranched or branched alcohols. In addition, linear, cyclic or branched hydrocarbons, with or without substituents, with or without double bonds. In addition, vegetable oils, carbonates with unbranched or branched alcohols, carbonates with unbranched or branched polyhydric alcohols, carbonates with linear or branched fatty alcohols, more preferably linear, cyclic or branched hydrocarbons, with or without substituents, with or without double bonds. In addition, carbonates with unbranched or branched alcohols, carbonates with unbranched or branched polyhydric alcohols, carbonates with linear or branched fatty alcohols. Esters of branched fatty acids with unbranched or branched polyhydric alcohols.

It is preferred according to the invention if component B), the cosmetic oil, contains no silicone oils.

It is also especially advantageous, and hence preferred, if the inventive compositions contain no silicone-containing components whatsoever. They are therefore silicone-free. In the context of the silicone-free inventive compositions, it is preferred according to the invention if component A) is selected from ester quats, wherein the ester quats described above as being preferably included are also accordingly preferably included in the context of the silicone-free inventive compositions.

According to the invention, component C) is at least one nonionic surfactant.

Preferred nonionic surfactants are selected from the group comprising, preferably consisting of:

addition products of ethylene oxide and/or propylene oxide onto linear fatty alcohols, fatty acids, fatty acid amides, fatty amines and alkylphenols,
glycerol monoesters and diesters and sorbitan monoesters and diesters of saturated and unsaturated fatty acids and the addition products thereof with ethylene oxide,
alkyl mono- and -oligoglycosides and the addition products thereof with ethylene oxide, addition products of ethylene oxide with castor oil and/or hydrogenated castor oil,
partial esters based on linear, branched, unsaturated and/or saturated fatty acids, ricinoleic acid, 12-hydroxystearic acid, glycerol, polyglycerol, pentaerythritol, dipentaerythritol and sugar alcohols (e.g. sorbitol), alkylglucosides (e.g. methylglucoside, butylglucoside, laurylglucoside) and also polyglucosides (e.g. cellulose), mono-, di- and trialkyl phosphates and also mono-, di- and/or tri-PEG-alkyl phosphates and their salts, citric esters such as glyceryl stearate citrate, glyceryl oleate citrate and dilauryl citrate, for example, and also glyceryl caprylate, polyglyceryl caprylates and polyglyceryl caprates,
and also alkoxylated triglycerides, mixed ethers and mixed formals, optionally partially oxidized alkyloligoglycosides or alkenyloligoglycosides or glucuronic acid derivatives, fatty acid N-alkylglucamides, protein hydrolysates (especially wheat-based plant products), polyol fatty acid esters, sugar esters, polysorbates and amine oxides, and mixtures of these surfactants.

Where the nonionic surfactants contain polyglycol ether chains, they may have a conventional homologue distribution, but preferably a narrowed homologue distribution.

More preferred nonionic surfactants as component C) are polyglycerol esters. The term “polyglycerol esters” in connection with the present invention includes partial polyglycerol esters, and hence compounds in which not all the hydroxyl groups have been esterified.

Preferably in accordance with the invention, the polyglycerol ester of component C) is selected from those of the general formula IV

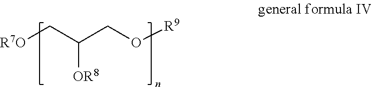

where
n=2 to 16, preferably 3-14, more preferably 4-11, and
R⁷, R⁸, R⁹═ are independently the same or different and are selected from H and saturated or unsaturated, linear or branched acyl radical having 4-36 carbon atoms and optionally containing at least one hydroxyl group, especially selected from the acyl radicals of natural fatty acids.

Polyglycerol esters of the general formula IV preferably used according to the invention are characterized in that the weight ratio of the polyglyceryl radical to the sum total of the acyl radicals R⁷; R⁸ and R⁹ is 90:10 to 45:55, preferably 88:12 to 50:50.

R⁷ is preferably H and the R⁸ and R⁹ radicals are preferably H or acyl radicals of natural fatty acids. R⁸ and R⁹ may also represent mixtures of such acyl radicals, particularly technical mixtures, for example coconut fatty acid cuts.

For R⁸ and R⁹, it is especially preferred that, based on all the R⁸ and R⁹ radicals in the polyglycerol ester, at least 50 mol %, preferably at least 75 mol %, of the acyl radicals R⁹ are selected from capryloyl, caproyl and lauroyl radicals.

Those skilled in the art are aware that the polyglycerol base skeleton present in the general formula IV, owing to its polymeric nature, is a random mixture of different compounds. Polyglycerol may have ether bonds formed between two primary, one primary and one secondary or else two secondary positions of the glycerol monomers. For this reason, the polyglycerol base skeleton does not usually consist exclusively of linearly linked glycerol units, but may also comprise branches and rings. For details see, e.g. “Original synthesis of linear, branched and cyclic oligoglycerol standards”, Cassel et al., J. Org. Chem., 2001, 875-896.

Structures of this kind are covered in the general formula IV which has been simplified in this respect.

The degree of polymerization n can be determined by determining the hydroxyl number of the polyglycerol used for synthesis of the inventive ester, where the mean degree of polymerization n is related to the hydroxyl number of the parent polyglycerol via the following equation:

$$n = \frac{\frac{2000 \cdot M ( KOH )}{OHN} - M ( water )}{[ [ M ( Glycerol ) - M ( water ) ] - \frac{1000 \cdot M ( KOH )}{OHN} ]}$$

where M=molar mass; OHN=hydroxyl number of the free polyglycerol.

Alternatively, the degree of polymerization n can also be determined by determining the hydroxyl number of the polyglycerol obtained after complete ester hydrolysis.

Suitable methods for determining the hydroxyl number are particularly those according to DGF C-V 17 a (53), Ph. Eur. 2.5.3 Method A and DIN 53240.

A preferred composition of the present invention is characterized in that it contains

D) a nonaqueous solvent selected from the group comprising, preferably consisting of: hydrotropes, for example from the group of the aliphatic alcohols, such as ethanol, propanol or propane-1,3-diol, cyclic carbonates such as ethylene carbonate, propylene carbonate, glycerol carbonate, esters of mono- or polycarboxylic acids such as ethyl acetate, ethyl lactate, glycerol, isopropyl alcohol, dipropylene glycol, glycol ethers (available, for example, under the DOWANOL® name from Dow Chemicals) and polyols. Polyols which are contemplated here may possess 2 to 15 carbon atoms and at least two hydroxyl groups. Typical examples are: glycerol, alkylene glycols, such as for example ethylene glycol, diethylene glycol, propylene glycol, butylene glycol, pentylene glycol, hexylene glycol, 1,2-propanediol, 1,2-butanediol, 1,2-pentanediol, 1,2-hexanediol and also polyethylene glycol or polypropylene glycol, polyhydroxycarboxylic acids, butyl diglycol and mixtures of these solvents.

Especially preferably, the nonaqueous solvent D) is selected from the group consisting of glycerol, glycol, 1,2-propylene glycol, 1,3-propylene glycol, butylene glycol and dipropylene glycol.

A further preferred composition of the present invention is characterized in that it comprises an amphoteric surfactant as additional component F), especially up to 10% based on the total composition. Typical examples of amphoteric surfactants are amphoacetates, amphopropionates, alkyl betaines, alkylamidobetaines, aminopropionates, aminoglycinates, imidazolinium betaines and sulfobetaines such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and also cocoacylaminoethylhydroxyethylcarboxymethyl glycinate. An especially preferred amphoteric surfactant is that known under the INCI name Cocamidopropyl Betaine.

The compositions of the present invention, especially the inventive microemulsions, can advantageously be used for the production of care and cleaning formulations, especially for skin and skin appendages, such as, for example, conditioning agents for hair, these being preferably selected from the group of cosmetic, cleaning and caring formulations. The present invention therefore also provides such uses.

The term “care formulation” is understood here as meaning a formulation which satisfies the purpose of retaining an article in its original form, of reducing or avoiding the effects of external influences (e.g. time, light, temperature, pressure, soiling, chemical reaction with other reactive compounds that come into contact with the article) such as, for example, ageing, soiling, material fatigue, bleaching, or even of improving desired positive properties of the article. For the latter point, for instance, improved shine of the hair or increased elasticity of the article in question may be mentioned.

In this connection, the care and cleaning formulations may be cosmetic, pharmaceutical or dermatological formulations, e.g. for treating the hair in the form of hair shampoos, 2-in-1 shampoos, liquid soaps, hair rinses, perming-fixing solutions, hair coloring shampoos, hair-setting compositions, hair treatments, hair arranging agents, hair styling preparations, blow-dry lotions, mousses, leave-in conditioners, hair smoothing agents, shine improving agents, and agents for coloring the hair.

This invention further provides for the use of the inventive microemulsions as conditioner for hair treatment agents and hair after-treatment agents, and also as agents for improving the structure of the hair.

The use according to the invention may take place, for example, in the form of a process according to the invention for the production of care and cleaning formulations, characterized by the process steps of:

1) providing an inventive composition, especially an inventive microemulsion,
2) mixing with an aqueous phase, preferably comprising at least one preservative and/or at least one perfume.

The term “preservative” in the context of the present invention is to be understood to mean an agent which effects preservation with respect to microbial, particularly bacterial, growth.

The present invention therefore additionally also provides the care and cleaning formulations, especially for skin and skin appendages, especially cosmetic formulations, these being preferably selected from the group of rinse-out or leave-in hair treatment agents and hair after-treatment agents, for example shampoos with or without pronounced conditioner effect, 2-in-1 shampoos, rinses, hair treatments, hair masks, styling aids, styling compositions, blow-dry lotions, hair-setting compositions, perming agents, hair smoothing agents and agents for coloring the hair, especially conditioning agents and shampoos containing at least one inventive composition.

Cosmetic formulations more preferred according to the invention are themselves also substantially free from alkoxylated compounds.

A formulation preferred according to the invention comprises the inventive microemulsion in an amount of 0.1 wt % to 99 wt %, preferably in an amount of 0.5 wt % to 20 wt %, more preferably in an amount of 1.0 wt % to 10 wt %, where the percentages by weight refer the total formulation.

The formulation according to the invention, especially the cosmetic formulation, can comprise for example at least one additional component selected from the group of

emollients,
emulsifiers,
thickeners/viscosity regulators/stabilizers,
antioxidants,
hydrotropes (or polyols),
solids and fillers,
pearlescence additives,
deodorant and antiperspirant active ingredients,
insect repellents,
self-tanning agents,
preservatives,
conditioning agents,
perfumes,
dyes,
cosmetic active ingredients,
care additives,
superfatting agents.

Substances which can be used as exemplary representatives of the individual groups are known to those skilled in the art and can be found for example in EP2273966A1. This patent application is herewith incorporated as reference and thus forms part of the disclosure.

As regards further optional components and the amounts used of these components, reference is made expressly to the relevant handbooks known to those skilled in the art, for example K. Schrader, “Grundlagen und Rezepturen der Kosmetika [Cosmetics—fundamentals and formulations]”, 2nd edition, pages 329 to 341, Huthig Buch Verlag Heidelberg.

The amounts of the particular additives are guided by the intended use.

Typical guide formulations for the respective applications are known prior art and are contained for example in the brochures of the manufacturers of the particular basic materials and active ingredients. These existing formulations can generally be adopted unchanged. If necessary, the desired modifications can, however, be undertaken without complication by means of simple experiments for the purposes of adaptation and optimization.

The examples adduced hereinafter describe the present invention by way of example, without any intention that the invention, the scope of application of which is apparent from the entirety of the description and the claims, be restricted to the embodiments specified in the examples.

The following FIGURES are part of the examples:

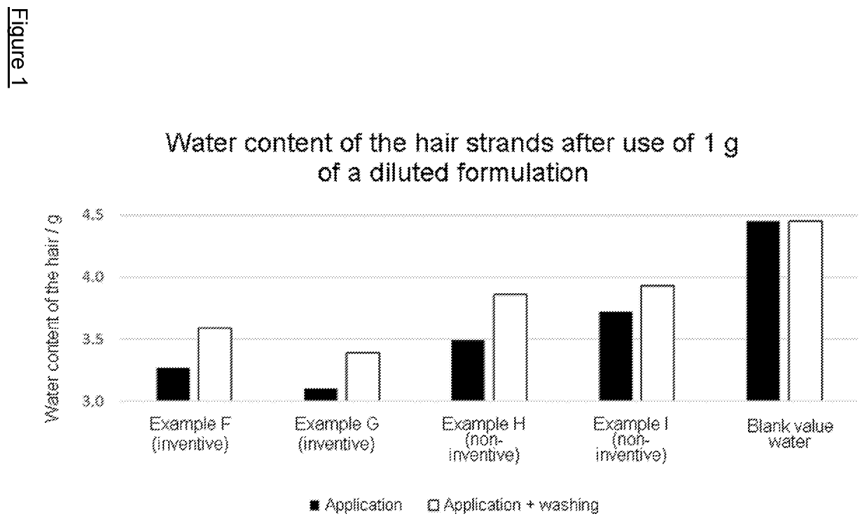

FIG. 1: Water content of the hair strands after use of 1 g of a diluted formulation

### EXAMPLES

### Example ME1: Inventive Microemulsion

25.0% of TEGOSOFT M (Isopropyl Myristate, Evonik Nutrition & Care GmbH), 12.5% of VARISOFT PATC (Palmitamidopropyltrimonium Chloride, Evonik Nutrition & Care GmbH), 10.0% of dipropylene glycol and 30.0% of TEGOSOFT PC 31 (Polyglyceryl-3 Caprate, Evonik Nutrition & Care GmbH) were mixed in 22.5% of water. A clear microemulsion is formed. The proportion of significantly hair-conditioning active substances (TEGOSOFT M and VARISOFT PATC) is 37.5%.

### Example ME2: Inventive Microemulsion

26.7% of TEGOSOFT M (Isopropyl Myristate, Evonik Nutrition & Care GmbH), 13.3% of VARISOFT EQ 100 (Bis-(Isostearoyl/Oleoyl Isopropyl) Dimonium Methosulfate, Evonik Nutrition & Care GmbH), 24.0% of dipropylene glycol and 26.7% of TEGOSOFT PC 41 (Polyglyceryl-4 Caprate, Evonik Nutrition & Care GmbH) were mixed in 9.3% of water. A clear microemulsion is formed. The proportion of significantly hair-conditioning active substances (TEGOSOFT M and VARISOFT EQ 100) is 40%.

### Example ME3: Inventive Microemulsion

25.0% of TEGOSOFT M (Isopropyl Myristate, Evonik Nutrition & Care GmbH), 2.5% of VARISOFT PATC (Palmitamidopropyltrimonium Chloride, Evonik Nutrition & Care GmbH), 10.0% of propylene glycol, 20% of TEGOSOFT PC 31 (Polyglyceryl-3 Caprate, Evonik Nutrition & Care GmbH) and 15.0% of TEGOSOFT PC 41 (Polyglyceryl-4 Caprate, Evonik Nutrition & Care GmbH) were mixed in 27.5% of water. A clear microemulsion is formed. The proportion of significantly hair-conditioning active substances (TEGOSOFT M and VARISOFT PATC) is 27.5%.

### Example ME4: Noninventive Microemulsion

Plantasil Micro (Dicaprylyl Ether (and) Decyl Glucoside (and) Glyceryl Oleate, BASF), with approximately 28% of hair-conditioning active constituents (Dicaprylyl Ether and Glyceryl Oleate).

### Example ME5: Noninventive Microemulsion

Lamesoft OD (Coco-Caprylate (and) Lauryl Glucoside (and) Glycerin (and) Polyglyceryl-2 Dipolyhydroxystearate (and) Polyglyceryl-3 Diisostearate, BASF).

### Example ME6: Inventive Microemulsion

23.9% of TEGOSOFT CT (Caprylic/Capric Triglyceride, Evonik Nutrition & Care GmbH), 21.0% of TEGOSOFT PC 31 (Polyglyceryl-3 Caprate, Evonik Nutrition & Care GmbH), 9.0% of VARISOFT EQ 100 (Bis-(Isostearoyl/Oleoyl Isopropyl) Dimonium Methosulfate, Evonik Nutrition & Care GmbH) and 18.0% of dipropylene glycol were mixed in 28.1% of water. A clear microemulsion is formed. The proportion of significantly hair-conditioning active substances (TEGOSOFT CT and VARISOFT EQ 100) is 32.9%.

### Example ME7: Inventive Microemulsion

21.0% of TEGOSOFT DEC (Diethylhexyl Carbonate, Evonik Nutrition & Care GmbH), 18.0% of TEGOSOFT PC 31 (Polyglyceryl-3 Caprate, Evonik Nutrition & Care GmbH), 8.0% of VARISOFT EQ 100 (Bis-(Isostearoyl/Oleoyl Isopropyl) Dimonium Methosulfate, Evonik Nutrition & Care GmbH) and 15.6% of dipropylene glycol were mixed in 37.4% of water. A clear microemulsion is formed. The proportion of significantly hair-conditioning active substances (TEGOSOFT DEC and VARISOFT EQ 100) is 29.0%.

### Example ME8: Inventive Microemulsion

19.8% of TEGOSOFT DEC (Diethylhexyl Carbonate, Evonik Nutrition & Care GmbH), 17.3% of TEGOSOFT PC 31 (Polyglyceryl-3 Caprate, Evonik Nutrition & Care GmbH), 5.0% of TEGO Betain F 50 (Cocamidopropyl Betaine, Evonik Nutrition & Care GmbH), 7.0% of VARISOFT EQ 100 (Bis-(Isostearoyl/Oleoyl Isopropyl) Dimonium Methosulfate, Evonik Nutrition & Care GmbH) and 14.0% of dipropylene glycol were mixed in 36.9% of water. A clear microemulsion is formed. The proportion of significantly hair-conditioning active substances (TEGOSOFT DEC and VARISOFT EQ 100) is 26.8%.

### Example ME9: Inventive Microemulsion

15.8% of TEGOSOFT OER (Oleyl Erucate, Evonik Nutrition & Care GmbH), 15.8% of TEGOSOFT M (Isopropyl Myristate, Evonik Nutrition & Care GmbH), 22.8% of TEGO Solve 61 (Polyglyceryl-6 Caprylate (and) Polyglyceryl-3 Cocoate (and) Polyglyceryl-4 Caprate (and) Polyglyceryl-6 Ricinoleate, Evonik Nutrition & Care GmbH), 22.8% of VARISOFT EQ 100 (Bis-(Isostearoyl/Oleoyl Isopropyl) Dimonium Methosulfate, Evonik Nutrition & Care GmbH) and 10.1% of dipropylene glycol were mixed in 12.7% of water. A clear microemulsion is formed. The proportion of significantly hair-conditioning active substances (TEGOSOFT OER, TEGOSOFT M und VARISOFT EQ 100) is 54.4%.

### Example ME10: Inventive Microemulsion

15.8% of TEGOSOFT OER (Oleyl Erucate, Evonik Nutrition & Care GmbH), 15.8% of TEGOSOFT M (Isopropyl Myristate, Evonik Nutrition & Care GmbH), 22.8% of TEGOSOFT PC 41 (Polyglyceryl-4 Caprate, Evonik Nutrition & Care GmbH), 22.8% of VARISOFT EQ 100 (Bis-(Isostearoyl/Oleoyl Isopropyl) Dimonium Methosulfate, Evonik Nutrition & Care GmbH) and 10.1% of dipropylene glycol were mixed in 12.7% of water. A clear microemulsion is formed. The proportion of significantly hair-conditioning active substances (TEGOSOFT OER, TEGOSOFT M and VARISOFT EQ 100) is 54.4%.

### Example ME11: Inventive Microemulsion

25.0% of TEGOSOFT P (Isopropyl Palmitate, Evonik Nutrition & Care GmbH), 18.0% of TEGOSOFT PC 41 (Polyglyceryl-4 Caprate, Evonik Nutrition & Care GmbH), 18.0% of VARISOFT EQ 100 (Bis-(Isostearoyl/Oleoyl Isopropyl) Dimonium Methosulfate, Evonik Nutrition & Care GmbH) and 8.0% of dipropylene glycol were mixed in 31.0% of water. A clear microemulsion is formed. The proportion of significantly hair-conditioning active substances (TEGOSOFT P and VARISOFT EQ 100) is 43%.

### Example ME12: Inventive Microemulsion

8.3% of TEGOSOFT OER (Oleyl Erucate, Evonik Nutrition & Care GmbH), 22.9% of TEGOSOFT P (Isopropyl Palmitate, Evonik Nutrition & Care GmbH), 16.5% of TEGOSOFT PC 41 (Polyglyceryl-4 Caprate, Evonik Nutrition & Care GmbH), 16.5% of VARISOFT EQ 100 (Bis-(Isostearoyl/Oleoyl Isopropyl) Dimonium Methosulfate, Evonik Nutrition & Care GmbH) and 7.3% of dipropylene glycol were mixed in 28.5% of water. A clear microemulsion is formed. The proportion of significantly hair-conditioning active substances (TEGOSOFT OER, TEGOSOFT P and VARISOFT EQ 100) is 47.7%.

### Example ME13: Inventive Microemulsion

6.3% of TEGOSOFT OER (Oleyl Erucate, Evonik Nutrition & Care GmbH), 18.7% of TEGOSOFT P (Isopropyl Palmitate, Evonik Nutrition & Care GmbH), 18.0% of TEGO Solve 61 (Polyglyceryl-6 Caprylate (and) Polyglyceryl-3 Cocoate (and) Polyglyceryl-4 Caprate (and) Polyglyceryl-6 Ricinoleate, Evonik Nutrition & Care GmbH), 18.0% of VARISOFT EQ 100 (Bis-(Isostearoyl/Oleoyl Isopropyl) Dimonium Methosulfate, Evonik Nutrition & Care GmbH) and 8.0% of dipropylene glycol were mixed in 31.0% of water. A clear microemulsion is formed. The proportion of significantly hair-conditioning active substances (TEGOSOFT OER, TEGOSOFT P and VARISOFT EQ 100) is 43%.

### Example ME14: Inventive Microemulsion

15.8% of TEGOSOFT OER (Oleyl Erucate, Evonik Nutrition & Care GmbH), 15.8% of TEGOSOFT P (Isopropyl Palmitate, Evonik Nutrition & Care GmbH), 11.4% of TEGO Solve 61 (Polyglyceryl-6 Caprylate (and) Polyglyceryl-3 Cocoate (and) Polyglyceryl-4 Caprate (and) Polyglyceryl-6 Ricinoleate, Evonik Nutrition & Care GmbH), 11.4% of TEGOSOFT PC 41 (Polyglyceryl-4 Caprate, Evonik Nutrition & Care GmbH), 22.8% of VARISOFT EQ 100 (Bis-(Isostearoyl/Oleoyl Isopropyl) Dimonium Methosulfate, Evonik Nutrition & Care GmbH) and 10.1% of dipropylene glycol were mixed in 12.7% of water. A clear microemulsion is formed. The proportion of significantly hair-conditioning active substances (TEGOSOFT OER, TEGOSOFT P and VARISOFT EQ 100) is 54.4%.

### Example ME15: Inventive Microemulsion

25.0% of TEGOSOFT DEC (Diethylhexyl Carbonate, Evonik Nutrition & Care GmbH), 30.0% of TEGOSOFT PC 31 (Polyglyceryl-3 Caprate, Evonik Nutrition & Care GmbH), 5.0% of VARISOFT PATC (Palmitamidopropyltrimonium Chloride, Evonik Nutrition & Care GmbH) and 15.0% of propylene glycol were mixed in 25.0% of water. A clear microemulsion is formed. The proportion of significantly hair-conditioning active substances (TEGOSOFT DEC and VARISOFT PATC) is 30.0%.

### Example ME16: Inventive Microemulsion

18.4% of TEGOSOFT AC (Isoamyl Cocoate, Evonik Nutrition & Care GmbH), 20.1% of TEGOSOFT PC 41 (Polyglyceryl-4 Caprate, Evonik Nutrition & Care GmbH), 16.9% of VARISOFT EQ 100 (Bis-(Isostearoyl/Oleoyl Isopropyl) Dimonium Methosulfate, Evonik Nutrition & Care GmbH) 1.0% of VARISOFT 300 (Cetrimonium Chloride, Evonik Nutrition & Care GmbH), 1.0% of REWOTERIC AM C (Sodium Cocoamphoacetate, Evonik Nutrition & Care GmbH) and 13.8% of dipropylene glycol were mixed in 28.8% of water. A clear microemulsion is formed. The proportion of significantly hair-conditioning active substances (TEGOSOFT AC, VARISOFT 300 and VARISOFT EQ 100) is 36.3%.

### Example ME17: Inventive Microemulsion

17.8% of TEGOSOFT AC (Isoamyl Cocoate, Evonik Nutrition & Care GmbH), 19.3% of TEGOSOFT PC 41 (Polyglyceryl-4 Caprate, Evonik Nutrition & Care GmbH), 16.5% of VARISOFT EQ 100 (Bis-(Isostearoyl/Oleoyl Isopropyl) Dimonium Methosulfate, Evonik Nutrition & Care GmbH) 1.0% of VARISOFT 300 (Cetrimonium Chloride, Evonik Nutrition & Care GmbH), 2.0% of ANTIL Soft SC (Sorbitan Sesquicaprylate, Evonik Nutrition & Care GmbH) and 16.5% of dipropylene glycol were mixed in 26.9% of water. A clear microemulsion is formed. The proportion of significantly hair-conditioning active substances (TEGOSOFT AC, VARISOFT 300 and VARISOFT EQ 100) is 35.3%.

### Example ME18: Inventive Microemulsion

25.5% of TEGOSOFT M (Isopropyl Myristate, Evonik Nutrition & Care GmbH), 4.9% of TEGOSOFT OER (Oleyl Erucate, Evonik Nutrition & Care GmbH), 12.6% of VARISOFT EQ 100 (Bis-(Isostearoyl/Oleoyl Isopropyl) Dimonium Methosulfate, Evonik Nutrition & Care GmbH), 22.8% of dipropylene glycol and 25.4% of TEGOSOFT PC 41 (Polyglyceryl-4 Caprate, Evonik Nutrition & Care GmbH) were mixed in 8.8% of water. A clear microemulsion is formed. The proportion of significantly hair-conditioning active substances (TEGOSOFT M, TEGOSOFT OER and VARISOFT EQ 100) is 43%.

### Example ME19: Inventive Microemulsion

25.3% of TEGOSOFT M (Isopropyl Myristate, Evonik Nutrition & Care GmbH), 4.8% of argan oil (Argania Spinosa Oil (Argania Spinosa Kernel Oil), DSM Nutritional Products Ltd.), 12.7% of VARISOFT EQ 100 (Bis-(Isostearoyl/Oleoyl Isopropyl) Dimonium Methosulfate, Evonik Nutrition & Care GmbH), 22.9% of dipropylene glycol and 25.4% of TEGOSOFT PC 41 (Polyglyceryl-4 Caprate, Evonik Nutrition & Care GmbH) were mixed in 8.9% of water. A clear microemulsion is formed. The proportion of significantly hair-conditioning active substances (TEGOSOFT M, argan oil and VARISOFT EQ 100) is 42.8%.

### Example ME20: Inventive Microemulsion

26.7% of TEGOSOFT M (Isopropyl Myristate, Evonik Nutrition & Care GmbH), 13.3% of VARISOFT EQ F 75 Pellets (70% Distearoylethyl Hydroxyethylmonium Methosulfate; 30% Cetearyl Alcohol, Evonik Nutrition & Care GmbH), 24% of dipropylene glycol and 26.7% of TEGOSOFT PC 41 (Polyglyceryl-4 Caprate, Evonik Nutrition & Care GmbH) were mixed in 9.3% of water. A clear microemulsion is formed. The proportion of significantly hair-conditioning active substances (TEGOSOFT M and Distearoylethyl Hydroxyethylmonium Methosulfate) is 36%.

### Example ME21: Inventive Microemulsion

30.0% of siloxane, a Polysiloxane Multi-T-Quat with N=350 having the following statistical formula: (R₂Me₂SiO_{1/2})₇ (Me₂SiO_{1/2})₃₃₈ (MeSiO_{3/2})₅, with

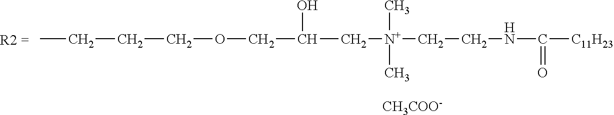

10.0% of propylene glycol, 10.0% of TEGOSOFT PC 31 (Polyglyceryl-3 Caprate, Evonik Nutrition & Care GmbH), 5.0% of TEGO Betain F 50 (Cocamidopropyl Betaine, 38% strength, Evonik Nutrition & Care GmbH), 2.4% VARISOFT PATC (Palmitamidopropyltrimonium Chloride, Evonik Nutrition & Care GmbH) were mixed in 42.6% of water. A clear microemulsion is formed. The proportion of significantly hair-conditioning active substances (siloxane and VARISOFT PATC) is 32.4%.

### Example ME22: Inventive Microemulsion

8% of Glyceryl Monooleate (TEGIN O V, Evonik), 25% of Dioctylether (Cetiol OE, BASF), 10% of Hexadecyl Hexadecanoate (Cutinal CP, BASF), 20.4% of C12-16 fatty alcohol 1,4-glucoside (50% in H₂O)(Plantacar 1200 UP, BASF), 13.6% of C8-16 fatty alcohol 1,4-glucoside (50% in H₂O)(Plantacare 2000 UP, BASF), 5.0% of Trimethylhexadecylammonium Chloride (CTAC) (Aldrich-Merck), 1.0% of Benzoic acid, 2.0% of Citric acid were mixed in 15.0% of water. A clear microemulsion is formed. The proportion of significantly hair-conditioning active substances (Dioctylether, Hexadecyl Hexadecanoate and Trimethylhexadecylammonium Chloride) is 40%.

### Application-Related Properties:

Formulation constituents are named in the compositions which follow in the form of the commonly acknowledged INCI nomenclature using the English terms. All concentrations in the application examples are given in percent by weight.

Removal of the Odor of Frying from Hair, by Means of Moist Hair Wipes

### Formulations Tested:

### Example A (not According to the Invention):

0.67% of VARISOFT EQ 100 and 1.33% of TEGOSOFT M were stirred into water. A dispersion was formed, which separated after standing for a short time. This means that a homogeneous formulation and hence a homogeneous application to the moist wipes was not possible.

### Example B (Noninventive):

1.33% of TEGOSOFT M were stirred into water. A dispersion was formed, which separated after standing for a short time. This means that a homogeneous formulation and application to the moist wipes was not possible.

### Example C (Noninventive)

0.67% of VARISOFT EQ 100 was dispersed in water. A slightly cloudy dispersion was formed.

### Example D (Inventive):

5% of example ME2 was dispersed in water and a homogeneous, very slightly opaque solution was formed.

### Example E (Noninventive):

10% of Plantasil Micro (example ME4) was stirred into water. A homogeneous, clear solution was formed.

### Treatment of the Hair:

For the application-related assessment, hair tresses (4 g of Caucasian hair, Kerling, Germany) were predamaged by means of a bleach treatment in a standardized way. For this, customary hairstyling products were used. The test procedure, the base materials used and the details of the assessment criteria are described in DE 103 27 871.

The hair strands were pre-washed with an SLES solution (12% of Sodium Laureth Sulfate in water) and dried overnight in a climate-controlled chamber at 50% humidity and 22° C. A deep-fat fryer (Tristar FR-6935) containing 2 l of frying oil (Palmin) was set to 170° C. and 750 g of chips (Mc Cain, 1-2-3-Frites Original) were fried for 10 minutes. During the frying process, the hair strands were hung at a distance of 20 cm above the deep-fat fryer, such that they absorbed the odor of frying.

### Moist Wipes:

Moist wipes (Sontara Style 8838, Woodpulp/Polyester, 20×20 cm, 50.8 g/m², American FlexPack, Inc., Green Bay, Wis., USA) were sprinkled with 5 ml of the formulation solution and stored lying horizontally for 30 min at 25° C. in a closed airtight bag in order to ensure uniform distribution of the ingredients on the nonwoven fabric.

Subsequently, the hair strands were wiped three times from top to bottom with the moist wipe (the hair tress was guided within the moist wipe while doing so). The hair strands were dried for 1 h in a climate-controlled chamber (22° C., 50% humidity) and subsequently evaluated in a panel test.

### Panel Test:

The hair strands were evaluated on a scale of 1 to 5 (5=best) in terms of odor, feel (softness and silkiness of the hair) and appearance (non-greasiness) by a trained panel of 10 people. The mean values from the panel evaluations obtained are given in the following table. 5 hair strands were evaluated for each formulation.

| | Blank value | Example C | | Example E |
|---|---|---|---|---|
| | (demin. | (non- | Example D | (non- |
| | water) | inventive) | (inventive) | inventive) |
| Odor | 1.0 | 3.0 | 3.9 | 2.2 |
| Feel | 1.4 | 4.2 | 5.0 | 3.1 |
| Appearance | 1.0 | 3.1 | 4.1 | 3.3 |

It can be clearly seen that the inventive formulation D (with the microemulsion ME2 with VARISOFT EQ 100 and TEGOSOFT M) gives better panel results for all three evaluation criteria than the noninventive formulation C (only with VARISOFT EQ 100) and the noninventive formulation E with the commercially available microemulsion. In particular, the very good result in terms of odor removal is surprising in this case.

### Protection Against the Development of Odors on Hair

Hair strands, which were pre-washed as described above with an SLES solution, were treated with the moist wipes from application example 1 as described in said example, and subsequently hung over a deep-fat fryer as in example 1. Following the frying process, the odor of the hair was directly evaluated.

| | Blank value, | Example C | Example D | Example E |
|---|---|---|---|---|
| | water | (noninventive) | (inventive) | (noninventive) |
| Odor | 1.0 | 1.7 | 2.8 | 1.5 |

The result shows that the inventive formulation D also exerted a significantly protective effect on the hair in terms of the development of frying odor.

### Rapid Drying of Hair

Hair tresses (4 g of Caucasian hair, Kerling, Germany), which were predamaged by means of a bleach treatment in a standardized way, were pre-washed with a Sodium Laureth Sulfate/Cocamidopropyl Betaine solution (9%: 3%) and dried overnight in a climate-controlled chamber at 50% humidity and 22° C., and weighed the next day (m₁).

For the measurement, the strands were wetted for ten seconds with water (38° C.) and once again pre-washed with 1 g of the Sodium Laureth Sulfate/Cocamidopropyl Betaine solution (9%: 3%), by the surfactant solution being massaged in for 1 minute. The hair was subsequently rinsed for 1 minute with water (38° C.).

The wet hair was laid in a dish and the formulations to be tested were sprayed from an atomizer in diluted form thereon. 1 g of the diluted formulation was sprayed on; the tresses were regularly turned in the receptacle in order to achieve uniform coverage. The tresses were then left for 1 minute, and excess water was uniformly squeezed out twice between two fingers, from top to bottom. The hair strands were weighed again (m₂).

The hair was then washed again with 1 g of the Sodium Laureth Sulfate/Cocamidopropyl Betaine solution (9%: 3%) for 1 minute, rinsed with water for 1 minute and left to drip for 1 minute, without the hair being treated again with the formulations. The hair was squeezed out as described and weighed again (m₃).

10 hair tresses were tested per formulation, and the mean value was determined.

The water content of the hair after applying the formulations is calculated from the difference m₂-m₁ and describes the effect of applying the formulation on the water content of the hair.

The water content of the hair after application and washing the hair again is calculated from the difference m₃-m₁ and shows if the formulation still exerts an effect on the water content even after washing again.

### Example F (Inventive):

1.25% of example ME2 in water

### Example G (Inventive):

2.5% of example ME2 in water

### Example H (Noninventive):

2.5% of Plantasil Micro (example ME4; Dicaprylyl Ether (and) Decyl Glucoside (and) Glyceryl Oleate, BASF) in water

### Example I (Noninventive):

2.5% of Lamesoft OD (example ME5; Coco-Caprylate (and) Lauryl Glucoside (and) Glycerol (and) Polyglyceryl-2 Dipolyhydroxystearate (and) Polyglyceryl-3 Diisostearate, BASF) in water

As shown in FIG. 1, the formulations of example F and G with the inventive microemulsion of example ME2 surprisingly showed a significantly lower water uptake than the comparative examples. This can be measured both directly after use of the respective aqueous formulation and also after a subsequent wash with a surfactant formulation. The hair can therefore be dried more rapidly after using the inventive formulations.

### Testing the Conditioning of Hair by Means of Sensory Testing—Shampoo Application 1

For the application-related assessment of the conditioning of hair, the inventive microemulsions, examples ME1, ME2 and ME3, and also the comparative example, ME4, Plantasil Micro, were tested in a simple cosmetic shampoo formulation (table 1). In each case approximately 1% of active substance (i.e. the sum total of all primarily conditioning active substances) was used.

TABLE 1
Shampoo formulations for testing the hair conditioning properties.

| Formulation examples | 0a | 1a | 2a | 3a | C4a |
|---|---|---|---|---|---|
| Texapon NSO, 28% strength, | 32% | 32% | 32% | 32% | 32% |
| BASF, (INCI: Sodium | | | | | |
| Laureth Sulfate) | | | | | |
| TEGO Betain F 50, 38% | 8% | 8% | 8% | 8% | 8% |
| strength, Evonik Nutrition & | | | | | |
| Care GmbH, (INCI: | | | | | |
| Cocamidopropyl Betaine) | | | | | |
| UCARE Polymer JR-400, | 0.3% | 0.3% | 0.3% | 0.3% | 0.3% |
| Dow Chemicals, (INCI: | | | | | |
| Polyquaternium-10) | | | | | |
| ANTIL 171, Evonik | 2.5% | 2.5% | 2.5% | 2.5% | 2.5% |
| Nutrition & Care GmbH, | | | | | |
| (INCI: PEG-18 Glyceryl | | | | | |
| Oleate/Cocoate) | | | | | |
| NaCl | 0.2% | 0.2% | 0.2% | 0.2% | 0.2% |
| Water, demineralized | to 100.0% | | | | |
| Citric acid (10% solution in | to pH 5.5 ± 0.3 | | | | |
| water) | | | | | |
| Microemulsion example | — | 2.7 | — | — | — |
| ME1 (inventive) | | | | | |
| Microemulsion example | — | — | 2.5 | — | — |
| ME2 (inventive) | | | | | |
| Microemulsion example | — | — | — | 3.6 | — |
| ME3 (inventive) | | | | | |
| Comparative example | — | — | — | — | 3.6 |
| ME4 - Plantasil | | | | | |
| Micro (noninventive) | | | | | |
| Viscosity (Brookfield, | 6100 | 4000 | 3400 | 3600 | 40 |
| 25° C., in mPa · s) | | | | | |

It is noteworthy that, with the inventive formulations 1a, 2a and 3a, unlike the comparative formulation C4a, surprisingly significantly higher viscosities in the shampoo can be reached.

The hair tresses that were predamaged by means of bleaching as already described above in the first application example (“Removal of the odor of frying from hair, by means of moist hair wipes”) were treated as follows with the shampoo formulations: The hair tresses were wetted under running warm water. The excess water was squeezed out gently by hand, and then the shampoo was applied and worked gently into the hair (1 ml/hair tress (2 g)). After a contact time of 1 min, the hair was rinsed for 1 min with warm water (38° C.). The excess water was again gently squeezed out by hand and the wet sensory analysis was carried out. Before the sensory assessment of the dry hair, the tresses were dried for at least 12 h in the air at 50% humidity and 25° C.

Assessment criteria: The sensory evaluations are made using grades awarded on a scale from 1 to 5, with 1 being the worst evaluation and 5 being the best evaluation. The individual test criteria each receive their own evaluation. The test criteria are as follows: Wet combability, wet feel, dry combability, dry feel, appearance/shine.

The results of the sensory assessment of the treatment of the hair strands, carried out as described above, are compared in the following table. The panel consisted of 5 trained people.

TABLE 2
Sensory results of the conditioning of hair
from shampoo formulation

| | Wet | | Dry | | |
|---|---|---|---|---|---|
| | comb- | Wet | comb- | Dry | |
| | ability | feel | ability | feel | Shine |
| Control formulation 0a | 3.1 | 2.8 | 4.0 | 3.8 | 3.0 |
| (placebo) | | | | | |
| Inventive formulation 1a | 4.0 | 4.0 | 4.1 | 4.0 | 3.4 |
| Inventive formulation 2a | 4.3 | 4.2 | 4.3 | 4.1 | 4.0 |
| Inventive formulation 3a | 3.8 | 3.6 | 4.1 | 3.9 | 3.5 |
| Comparative formulation | 3.5 | 3.5 | 4.0 | 3.7 | 3.1 |
| C4a (noninventive) | | | | | |

The results show that, surprisingly, particularly the inventive formulation 2a with the inventive microemulsion ME2 receives significantly better evaluations than the comparative formulation C4a with the comparative example ME4 according to the prior art. The good assessment of the shine properties of all inventive formulations is particularly noteworthy. The two other inventive formulations 1a and 3a with the inventive microemulsions ME1 and ME3, respectively, are also significantly better than the comparative formulation C4a.

### Testing the Conditioning of Hair by Means of Sensory Testing—Conditioner Application

For the application-related assessment of the conditioning of hair, the inventive microemulsion of example ME2 was also tested in a simple cosmetic conditioner formulation (table 3). For comparison, the two conditioning active ingredients from ME2, i.e. Isopropyl Myristate and Ester quat, were also individually (and not in the form of a microemulsion) incorporated into a formulation. In each case 1% of active substance (i.e. the sum total of all primarily conditioning active substances) was used.

TABLE 3
Conditioner formulations for testing the
hair conditioning properties.

| Formulation examples | 0b | 1b | C2b |
|---|---|---|---|
| TEGO Alkanol 1618, Evonik | 7% | 7% | 7% |
| Nutrition & Care GmbH, | | | |
| (INCI: Cetearyl Alcohol) | | | |
| TEGINACID C, Evonik | 1% | 1% | 1% |
| Nutrition & Care GmbH, | | | |
| (INCI: Ceteareth-25) | | | |
| Microemulsion example | — | 2.5% | — |
| ME2 (inventive) | | | |
| VARISOFT EQ 100, Evonik | — | — | 0.33% |
| Nutrition & Care GmbH, | | | |
| (INCI: Bis-(Isostearoyl/Oleoyl | | | |
| Isopropyl) Dimonium | | | |
| Methosulfate) | | | |
| TEGOSOFT M, Evonik | — | — | 0.67% |
| Nutrition & Care GmbH, | | | |
| (INCI: Isopropyl Myristate) | | | |
| Water, demineralized | to 100.00% | | |
| Citric acid (10% solution in | to pH 4.0 ± 0.2 | | |
| water) | | | |
| Viscosity (Brookfield, 25° C., | 14 000 | 13 900 | 6000 |
| in mPa · s) | | | |

The inventive formulation 1b has surprisingly, unlike the comparative formulation C2b, a significantly higher viscosity in the conditioner (despite the additional constituents in the microemulsion).

The hair tresses that were predamaged by means of bleaching and prewashed as already described above in the first application example were treated as follows with the conditioner formulations: The hair tresses were wetted under running warm water. The excess water was squeezed out gently by hand, and then the conditioner was applied and worked gently into the hair (1 ml/hair tress (2 g)). After a contact time of 1 min, the hair was rinsed for 3 min with warm water (38° C.). The excess water was again gently squeezed out by hand and the wet sensory analysis was carried out.

Assessment criteria: The sensory evaluations are made using grades awarded on a scale from 1 to 5, with 1 being the worst evaluation and 5 being the best evaluation. The individual test criteria each receive their own evaluation. The test criteria are as follows: Detanglability of the hair, wet combability, wet feel.

The results of the sensory assessment of the treatment of the hair strands, carried out as described above, are compared in the following table 4. The panel consisted of 4 trained people.

TABLE 4
Sensory results of the conditioning of hair
from conditioner formulation

| | | Wet | Wet |
|---|---|---|---|
| | Detanglability | combability | feel |
| Control formulation | 2.0 | 1.8 | 1.6 |
| 0b (placebo) | | | |
| Inventive | 3.6 | 3.8 | 4.2 |
| formulation 1b | | | |
| Comparative | 3.3 | 3.3 | 3.5 |
| formulation C2b | | | |
| (noninventive) | | | |

The results show that, surprisingly, the inventive formulation 1b with the inventive microemulsion ME2 receives very much better evaluations than the comparative formulation C2b, in which the conditioning active substances were incorporated separately.

### Testing the Conditioning of Hair by Means of Sensory Testing Shampoo Application 2

For the application-related assessment of the conditioning of hair, the inventive microemulsions, examples ME2, ME20, ME21 and ME22 were tested in a simple cosmetic shampoo formulation (table 5).

TABLE 5
Shampoo formulations for testing the
hair conditioning properties.

| Formulation examples | 0c | 1c | 2c | 3c | 4c |
|---|---|---|---|---|---|
| Texapon NSO, 28% | 32% | 32% | 32% | 32% | 32% |
| strength, BASF, (INCI: | | | | | |
| Sodium Laureth Sulfate) | | | | | |
| TEGO Betain F 50, 38% | 8% | 8% | 8% | 8% | 8% |
| strength, Evonik | | | | | |
| Nutrition & Care GmbH, | | | | | |
| (INCI: Cocamidopropyl | | | | | |
| Betaine) | | | | | |
| UCARE Polymer JR- | 0.3% | 0.3% | 0.3% | 0.3% | 0.3% |
| 400, Dow Chemicals, | | | | | |
| (INCI: Polyquaternium- | | | | | |
| 10) | | | | | |
| ANTIL 171, Evonik | 2.5% | 2.5% | 2.5% | 2.5% | 2.5% |
| Nutrition & Care GmbH, | | | | | |
| (INCI: PEG-18 Glyceryl | | | | | |
| Oleate/Cocoate) | | | | | |
| NaCl | 0.2% | 0.2% | 0.2% | 0.2% | 0.2% |
| Water, demineralized | to 100.0% | | | | |
| Citric acid (10% | to pH 5.5 ± 0.3 | | | | |
| solution in water) | | | | | |
| Microemulsion example | — | 2.8 | — | — | — |
| ME20 (inventive) | | | | | |
| Microemulsion example | — | — | 2.5 | — | — |
| ME2 (inventive) | | | | | |
| Microemulsion example | — | — | — | 2.5 | — |
| ME22 (inventive) | | | | | |
| Microemulsion example | — | — | — | — | 3.1 |
| ME21 (inventive) - | | | | | |
| Viscosity (Brookfield, | 6000 | 1500 | 2900 | 540 | 110 |
| 25° C., in mPa · s) | | | | | |

The testing of the formulations from table 5 was also carried out exactly as described above for table 2:

TABLE 6
Sensory results of the conditioning of hair
from shampoo formulation

| | Wet | | Dry | | |
|---|---|---|---|---|---|
| | comb- | Wet | comb- | Dry | |
| | ability | feel | ability | feel | Shine |
| Control | 3.0 | 3.0 | 4.0 | 3.8 | 3.2 |
| formulation | | | | | |
| 0c (placebo) | | | | | |
| Inventive | 4.0 | 4.0 | 4.2 | 4.0 | 3.6 |
| formulation | | | | | |
| 1c | | | | | |
| Inventive | 4.3 | 4.3 | 4.3 | 4.2 | 3.9 |
| formulation | | | | | |
| 2c | | | | | |
| Inventive | 3.6 | 3.5 | 4.0 | 3.9 | 3.3 |
| formulation | | | | | |
| 3c | | | | | |
| Inventive | 4.4 | 4.5 | 4.3 | 3.7 | 3.9 |
| formulation | | | | | |
| 4c | | | | | |

The results show that the inventive formulation 2c has similarly very good values to the formulation 4c that is additionally provided with silicone quat, and also significantly exceeds the silicone quat formulation, especially in terms of dry feel.

For the formulations 1c and 2c, the good viscosities stand out; the formulations thus require fewer rheological additives and thickeners in order to achieve a viscosity customary for shampoos; thus, they are very readily thickened.

### Further Formulation Examples:

The formulation examples given in the tables below show exemplary representatives of a large number of possible compositions according to the invention.

If the preparation of the formulation requires the separate preparation or mixing of formulation constituents beforehand, this is termed multiphase preparation.

If a two-phase preparation is required, the two phases are labelled A and B in the stated tables. In the case of three-phase processes, the three phases are called A, B and C. Unless otherwise indicated, the amounts in the tables below are amounts in wt %.

### Formulation Example 1) Clear Shampoo

| | |
|---|---|
| TEXAPON ® NSO, BASF, 28% | 32.00% |
| strength (INCI: Sodium Laureth | |
| Sulfate) | |
| Microemulsion ME3 | 2.00% |
| Perfume | 0.20% |
| TEGO ® Betain F 50, Evonik | 8.00% |
| Nutrition & Care GmbH, 38% | |
| strength (INCI: Cocamidopropyl | |
| Betaine) | |
| ANTIL ® 171 Evonik Nutrition & | 1.70% |
| Care GmbH, (INCI: PEG-18 | |
| Glyceryl Oleate/Cocoate) | |
| Water | to 100.00% |
| NaCl | 0.80% |
| Citric acid, 30% strength | q.s. to pH 5.5 |
| Preservative | q. s. |

### Formulation Example 2) Clear Conditioning Shampoo

| | |
|---|---|
| TEXAPON ® NSO, BASF, 28% | 35.00% |
| strength (INCI: Sodium Laureth | |
| Sulfate) | |
| ANTIL ® 200, Evonik Nutrition & | 1.50% |
| Care GmbH (INCI: PEG-200 | |
| Hydrogenated Glyceryl Palmate; | |
| PEG-7 Glyceryl Cocoate) | |
| Microemulsion ME1 | 2.00% |
| Perfume | 0.25% |
| Water | to 100.00% |
| Polymer JR 400, Amerchol, | 0.20% |
| (INCI : Polyquaternium-10) | |
| TEGO ® Betain F 50, Evonik | 8.00% |
| Nutrition & Care GmbH, 38% | |
| strength | |
| (INCI: Cocamidopropyl Betaine) | |
| NaCl | 1.50% |
| Citric acid, 30% strength | q.s. to pH 5.5 |
| Preservative | q.s. |

### Formulation Example 3) Clear Conditioning Shampoo

| | |
|---|---|
| TEXAPON ® NSO, BASF, 28% | 20.00% |
| strength (INCI: Sodium Laureth | |
| Sulfate) | |
| ANTIL ® 200, Evonik Nutrition & | 1.80% |
| Care GmbH, (INCI: PEG-200 | |
| Hydrogenated Glyceryl Palmate; | |
| PEG-7 Glyceryl Cocoate) | |
| ABIL ® Quat 3272, Evonik | 1.00% |
| Nutrition & Care GmbH, (INCI: | |
| Quaternium-80) | |
| Microemulsion ME2 | 2.50% |
| Perfume | 0.25% |
| Water | to 100.00% |
| Polymer JR 400, Amerchol | 0.30% |
| (INCI : Polyquaternium-10) | |
| TEGO ® Betain F 50, Evonik | 18.00% |
| Nutrition & Care GmbH, 38% | |
| strength (INCI: Cocamidopropyl | |
| Betaine) | |
| NaCl | 0.90% |
| Lactic acid, 80% strength | q.s. to pH 5.5 |
| Preservative | q. s. |

### Formulation Example 4) Clear Conditioning Shampoo

| | | |
|---|---|---|
| TEXAPON ® NSO, BASF, 28% strength | | 32.00% |
| (INCI: Sodium Laureth Sulfate) | | |
| ANTIL ® 500 Pellets, Evonik Nutrition & Care GmbH | | 1.00% |
| (INCI: PEG-200 Glyceryl Stearate) | | |
| ABIL ® B 8832, Evonik Nutrition & Care GmbH | | 0.50% |
| (INCI: Bis-PEG/PPG-20/20 Dimethicone) | | |
| Microemulsion ME6 | | 3.50% |
| Perfume | | 0.20% |
| Water | to | 100.00% |
| Polymer JR 400, Amerchol | | 0.20% |
| (INCI: Polyquaternium-10) | | |
| TEGO ® Betain F 50, Evonik | | 8.00% |
| Nutrition & Care GmbH, 38% strength | | |
| (INCI: Cocamidopropyl Betaine) | | |
| NaCl | | 0.80% |
| Citric acid, 30% strength | | q.s. to |
| | | pH 5.5 |
| Preservative | | q. s. |

### Formulation Example 5) Clear Conditioning Shampoo

| | | |
|---|---|---|
| TEXAPON ® NSO, BASF, 28% strength | | 32.00% |
| (INCI: Sodium Laureth Sulfate) | | |
| VARISOFT ® PATC, Evonik Nutrition & Care GmbH, | | 1.50% |
| (INCI: Palmitamidopropyltrimonium Chloride) | | |
| REWODERM ® LI S 80, Evonik Nutrition & Care | | 2.50% |
| GmbH, (INCI: PEG-200 Hydrogenated Glyceryl | | |
| Palmate; PEG-7 Glyceryl Cocoate) | | |
| Microemulsion ME13 | | 2.00% |
| Perfume | | 0.15% |
| Water | to | 100.00% |
| TEGO ® Cosmo C 100, Evonik Nutrition & Care GmbH, | | 1.00% |
| (INCI: Creatine) | | |
| Jaguar C-162, Solvay, (INCI: Hydroxypropyl | | 0.30% |
| Guar Hydroxypropyltrimonium Chloride) | | |
| TEGO ® Betain F 50, Evonik Nutrition & Care GmbH, | | 8.00% |
| 38% strength (INCI: Cocamidopropyl Betaine) | | |
| NaCl | | 1.50% |
| Citric acid, 30% strength | | q.s. to |
| | | pH 5.5 |
| Preservative | | q. s. |

### Formulation Example 6) Clear Conditioning Shampoo

| | | |
|---|---|---|
| Microemulsion ME16 | | 2.50% |
| Argan Oil, DSM Nutritional Products Ltd., | | 0.1% |
| (INCI: Argania Spinosa Oil (Argania Spinosa | | |
| Kernel Oil)) | | |
| TEXAPON ® NSO, BASF, 28% strength | | 32.00% |
| (INCI: Sodium Laureth Sulfate) | | |
| REWODERM ® LI S 80, Evonik Nutrition & Care | | 2.00% |
| GmbH, (INCI: PEG-200 Hydrogenated Glyceryl | | |
| Palmate; PEG-7 Glyceryl Cocoate) | | |
| Perfume | | 0.25% |
| Water | to | 100.00% |
| TEGO ® Cosmo C 100, Evonik Nutrition & | | 1.50% |
| Care GmbH, (INCI: Creatine) | | |
| Jaguar C-162, Solvay, (INCI: Hydroxypropyl | | 0.20% |
| Guar Hydroxypropyltrimonium Chloride) | | |
| TEGO ® Betain F 50, Evonik Nutrition & Care | | 8.00% |
| GmbH, 38% strength (INCI: Cocamidopropyl Betaine) | | |
| NaCl | | 2.50% |
| Citric acid, 30% strength | | q.s. to |
| | | pH 5.0 |
| Preservative | | q. s. |

### Formulation Example 7) Pearlized Shampoo

| | | |
|---|---|---|
| TEXAPON ® NSO, BASF, 28% strength | | 32.00% |
| (INCI: Sodium Laureth Sulfate) | | |
| Microemulsion ME19 | | 5.50% |
| Perfume | | 0.15% |
| Water | to | 100.00% |
| TEGO ® Betain F 50, Evonik Nutrition & | | 8.00% |
| Care GmbH, 38% strength | | |
| (INCI: Cocamidopropyl Betaine) | | |
| TEGO ® Pearl N 300, Evonik Nutrition & Care | | 2.00% |
| GmbH, (INCI: Glycol Distearate; Laureth-4; | | |
| Cocamidopropyl Betaine) | | |
| ANTIL ® 171 Evonik Nutrition & Care GmbH, | | 2.50% |
| (INCI: PEG-18 Glyceryl Oleate/Cocoate) | | |
| NaCl | | 0.90% |
| Citric acid, 30% strength | | q.s. to |
| | | pH 5.5 |
| Preservative | | q.s. |

### Formulation Example 8) Turbid Conditioning Shampoo

| | | |
|---|---|---|
| TEXAPON ® NSO, BASF, 28% strength (INCI: | | 32.00% |
| Sodium Laureth Sulfate) | | |
| ANTIL ® 200, Evonik Nutrition & Care GmbH, (INCI: | | 2.00% |
| PEG-200 Hydrogenated Glyceryl Palmate; PEG-7 | | |
| Glyceryl Cocoate) | | |
| Microemulsion ME18 | | 1.00% |
| Perfume | | 0.25% |
| Water | to | 100.00% |
| Polymer JR 400, Amerchol, (INCI: Polyquaternium-10) | | 0.40% |
| TEGO ® Betain F 50, Evonik Nutrition & Care GmbH, | | 8.00% |
| 38% strength (INCI: Cocamidopropyl Betaine) | | |
| DC1503 Fluid, Dow Corning, (INCI: Dimethicone, | | 1.00% |
| Dimethiconol) | | |
| TEGO ® Pearl N 300, Evonik Nutrition & Care | | 2.00% |
| GmbH, (INCI: Glycol Distearate; Laureth-4; | | |
| Cocamidopropyl Betaine) | | |
| NaCl | | 0.30% |
| Citric acid, 30% strength | | q.s. to |
| | | pH 5.5 |
| Preservative | | q. s. |

### Formulation Example 9) Bodycare Foam

| | | |
|---|---|---|
| TEXAPON ® NSO, BASF, 28% strength | | 15.00% |
| (INCI: Sodium Laureth Sulfate) | | |
| Perfume | | 0.30% |
| Microemulsion ME7 | | 1.50% |
| REWOTERIC ® AM C, Evonik Nutrition & Care GmbH, | | 8.00% |
| 32% strength (INCI: Sodium Cocoamphoacetate) | | |
| Water | to | 100.00% |
| TEGOCEL ® HPM 50, Evonik Nutrition & Care GmbH, | | 0.50% |
| (INCI: Hydroxypropyl Methylcellulose) | | |
| LACTIL ®, Evonik Nutrition & Care GmbH, | | 1.00% |
| (INCI: Sodium Lactate; Sodium PCA; Glycine; Fructose; | | |
| Urea; Niacinamide; Inositol; Sodium benzoate; | | |
| Lactic Acid) | | |
| Citric Acid Monohydrate | | 0.50% |
| Preservative | | q. s. |

### Formulation Example 10) Bodycare Product

| | | |
|---|---|---|
| TEXAPON ® NSO, BASF, 28% strength | | 30.00% |
| (INCI: Sodium Laureth Sulfate) | | |
| TEGOSOFT ® PC 31, Evonik Nutrition & Care GmbH, | | 0.50% |
| (INCI: Polyglyceryl-3 Caprate) | | |
| Microemulsion ME2 | | 4.50% |
| Perfume | | 0.30% |
| Water | to | 100.00% |
| TEGOCEL ® HPM 4000, Evonik Nutrition & | | 0.30% |
| Care GmbH, (INCI: Hydroxypropyl Methylcellulose) | | |
| Jaguar C-162, Solvay, (INCI: Hydroxypropyl Guar | | 0.20% |
| Hydroxypropyltrimonium Chloride) | | |
| REWOTERIC ® AM C, Evonik Nutrition & Care GmbH, | | 12.00% |
| 32% strength (INCI: Sodium Cocoamphoacetate) | | |
| Citric Acid Monohydrate | | 0.50% |
| REWODERM ® LI S 80, Evonik Nutrition & | | 2.00% |
| Care GmbH, (INCI: PEG-200 Hydrogenated Glyceryl | | |
| Palmate; PEG-7 Glyceryl Cocoate) | | |
| TEGO ® Pearl N 300, Evonik Nutrition & Care GmbH, | | 2.00% |
| (INCI: Glycol Distearate; Laureth-4; Cocamidopropyl | | |
| Betaine) | | |
| NaCl | | 1.00% |
| Preservative | | q.s. |

### Formulation Example 11) Bodycare Foam

| | | |
|---|---|---|
| TEXAPON ® NSO, BASF, 28% strength | | 14.30% |
| (INCI: Sodium Laureth Sulfate) | | |
| ANTIL ® 500 pellets, Evonik Nutrition & Care GmbH | | 0.70% |
| (INCI: PEG-200 Glyceryl Stearate) | | |
| Perfume | | 0.30% |
| Microemulsion ME3 | | 1.00% |
| REWOTERIC ® AM C, Evonik Nutrition & Care GmbH, | | 10.00% |
| 32% strength (INCI: Sodium Cocoamphoacetate) | | |
| NaCl | | 1.50% |
| Water | to | 100.00% |
| Merquat 550, Nalco, (INCI: Polyquaternium-7) | | 0.30% |
| LACTIL ®, Evonik Nutrition & Care GmbH, | | 0.50% |
| (INCI: Sodium Lactate; Sodium PCA; Glycine; Fructose; | | |
| Urea; Niacinamide; Inositol; Sodium benzoate; | | |
| Lactic Acid) | | |
| Citric acid, 30% strength | | q.s. to |
| | | pH 5.5 |

### Formulation Example 12) Mild Foam Bath

| | | |
|---|---|---|
| TEXAPON ® NSO, BASF, 28% strength | | 27.00% |
| (INCI: Sodium Laureth Sulfate) | | |
| REWOPOL ® SB FA 30, Evonik Nutrition & | | 12.00% |
| Care GmbH, 40% strength | | |
| (INCI: Disodium Laureth Sulfosuccinate) | | |
| TEGOSOFT ® LSE 65 K SOFT, Evonik Nutrition & | | 2.00% |
| Care GmbH, (INCI: Sucrose Cocoate) | | |
| Water | to | 100.00% |
| Jaguar C-162, Solvay, (INCI: Hydroxypropyl | | 0.15% |
| Guar Hydroxypropyltrimonium Chloride) | | |
| REWOTERIC ® AM C, Evonik Nutrition & Care GmbH, | | 12.00% |
| 32% strength (INCI: Sodium Cocoamphoacetate) | | |
| Microemulsion ME7 | | 1.50% |
| Citric acid, 30% strength | | 3.00% |
| ANTIL ® 171, Evonik Nutrition & Care GmbH, | | 1.75% |
| (INCI: PEG-18 Glyceryl Oleate/Cocoate) | | |
| NaCl | | 1.00% |
| TEGO ® Pearl N 300 Evonik Nutrition & Care GmbH | | 2.00% |
| (INCI: Glycol Distearate; Laureth-4; Cocamidopropyl | | |
| Betaine) | | |

### Formulation Example 13) Moisturizing Skin Cleanser

| | | | |
|---|---|---|---|
| A | TEXAPON ® NSO, BASF, 28% strength, | | 30.00% |
| | (INCI: Sodium Laureth Sulfate) | | |
| | Microemulsion ME2 | | 3.70% |
| | Perfume | | 0.30% |
| B | Water | to | 100.00% |
| | TEGOCEL ® fluid HPM 4000, Evonik | | 1.20% |
| | Nutrition & Care GmbH, (INCI: Hydroxypropyl | | |
| | Methylcellulose) TEGO ® Betain C 60, | | |
| | Evonik Nutrition & Care GmbH, 46% strength, | | 8.10% |
| | (INCI: Cocamidopropyl Betaine) | | |
| | TEGOSOFT ® APM, Evonik | | 1.00% |
| | Nutrition & Care GmbH, | | |
| | (INCI: PPG-3 Myristyl Ether) | | |
| | Cutina TS, BASF (INCI:PEG- 3 Distearate) | | 1.00% |
| | REWODERM ® LI S 80, Evonik | | 1.50% |
| | Nutrition & Care | | |
| | GmbH, (INCI: PEG-200 Hydrogenated | | |
| | Glyceryl Palmate; PEG-7 Glyceryl Cocoate) | | |
| | Preservative | | 0.60% |
| | Citric acid, 30% strength | | q.s. to |
| | | | pH 5.5 |

### Formulation Example 14) Shower Gel

| | | |
|---|---|---|
| TEXAPON ® NSO, BASF, 28% strength | | 15.00% |
| (INCI: Sodium Laureth Sulfate) | | |
| Microemulsion ME3 | | 1.50% |
| Microemulsion ME19 | | 1.00% |
| Perfume | | 0.30% |
| PGFAC-S, BASF, (INCI: Sodium cocoyl hydrolysed | | 1.00% |
| wheat protein glutamate) | | |
| REWOPOL SB CS 50 B, Evonik Nutrition & Care | | 7.50% |
| GmbH, 40% strength, (INCI: Disodium PEG-5 | | |
| Laurylcitrate Sulfosuccinate; Sodium Laureth Sulfate) | | |
| Water | to | 100.00% |
| TEGO ® Betain F 50, Evonik Nutrition & Care GmbH, | | 9.00% |
| 38% strength, (INCI: Cocamidopropyl Betaine) | | |
| TEGO ® Betain 810, Evonik Nutrition & Care GmbH, | | 4.00% |
| 38% strength, (INCI: Capryl/Capramidopropyl Betaine) | | |
| Merquat 550, Nalco, (INCI: Polyquaternium-7) | | 0.50% |
| ANTIL ® 200, Evonik Nutrition & Care GmbH, (INCI: | | 2.00% |
| PEG-200 Hydrogenated Glyceryl Palmate; PEG-7 | | |
| Glyceryl Cocoate) | | |
| NaCl | | 1.80% |
| Citric acid, 30% strength | | q.s. to |
| | | pH 5.2 |
| Preservative | | q. s. |

### Formulation Example 15) Body Cleanser

| | | | |
|---|---|---|---|
| A | TEXAPON ® NSO, BASF, 28% strength, | | 30.00% |
| | (INCI: Sodium Laureth Sulfate) | | |
| | Microemulsion ME7 | | 1.50% |
| | ABIL ® B 8832, Evonik Nutrition & Care GmbH, | | 0.30% |
| | (INCI: Bis-PEG/PPG-20/20 Dimethicone) | | |
| | Perfume | | 0.30% |
| B | Water | to | 100.00% |
| | TEGOCEL ® fluid HPM 4000, Evonik Nutrition & | | 1.20% |
| | Care GmbH, (INCI: Hydroxypropyl Methylcellulose) | | |
| | Jaguar C-162, Solvay, (INCI: Hydroxypropyl | | 0.15% |
| | guar hydroxypropyltrimonium chloride | | |
| | Citric Acid Monohydrate | | 0.50% |
| | REWOTERIC ® AM C, Evonik Nutrition & Care | | 10.00% |
| | GmbH, 32% strength, (INCI: Sodium | | |
| | Cocoamphoacetate) | | |
| | Cutina TS, BASF, (INCI: PEG- 3 Distearate) | | 2.00% |
| | REWODERM ® LI S 80, Evonik Nutrition & Care | | 2.60% |
| | GmbH, (INCI: PEG-200 Hydrogenated | | |
| | Glyceryl Palmate; PEG-7 Glyceryl Cocoate) | | |
| C | Preservative | | 0.60% |

### Formulation Example 16) Body Cleansing Foam

| | | |
|---|---|---|
| TEXAPON ® NSO, BASF, 28% strength | | 18.00% |
| (INCI: Sodium Laureth Sulfate) | | |
| Perfume | | 0.30% |
| Microemulsion ME18 | | 0.70% |
| Cropure Olive, Croda, (INCI: Olea Europaea (Olive) | | 0.10% |
| Fruit Oil) | | |
| REWOTERIC ® AM C, Evonik Nutrition & Care GmbH, | | 8.00% |
| 32% strength (INCI: Sodium Cocoamphoacetate) | | |
| TEGO ® Betain F 50, Evonik Nutrition & Care GmbH, | | 8.00% |
| 38% strength (INCI: Cocamidopropyl Betaine) | | |
| Water | to | 100.00% |
| Jaguar C-162, Solvay, (INCI: Hydroxypropyl | | 0.25% |
| Guar Hydroxypropyltrimonium Chloride) | | |
| ANTIL ® Soft SC, Evonik Nutrition & Care GmbH, | | 0.50% |
| (INCI: Sorbitan Sesquicaprylate) | | |
| ANTIL ® 120 Plus, Evonik Nutrition & Care GmbH, | | 1.50% |
| (INCI: PEG-120 Methyl Glucose Dioleate) | | |
| TEGO ® Cosmo C 100, Evonik Nutrition & Care GmbH, | | 1.00% |
| (INCI: Creatine) | | |
| NaCl | | 1.00% |
| Panthenol, BASF, (INCI: D- Panthenol) | | 0.20% |
| Citric Acid Monohydrate | | 0.50% |

### Formulation Example 17) Anti-Dandruff Shampoo

| | | |
|---|---|---|
| TEXAPON ® LS 35, BASF, 30% strength | | 24.00% |
| (INCI: Sodium Lauryl Sulfate) | | |
| TAGAT ® CH 40, Evonik Nutrition & | | 2.00% |
| Care GmbH, (INCI: PEG-40 Hydrogenated | | |
| Castor Oil) | | |
| TEGOSOFT ® GC, Evonik Nutrition & | | 1.00% |
| Care GmbH, (INCI: PEG-7 Glyceryl Cocoate) | | |
| Cropure Avocado, Croda, | | 0.15% |
| (INCI: Persea Gratissima (Avocado) Oil) | | |
| Microemulsion ME19 | | 2.00% |
| Perfume | | 0.20% |
| Water | to | 100.00% |
| Polymer JR 400, Amerchol, (INCI: Poly | | 0.20% |
| quaternium-10) | | |
| TEGO ® Betain F 50, Evonik Nutrition & Care | | 16.00% |
| GmbH, 38% strength (INCI: Cocamidopropyl | | |
| Betaine) | | |
| Hostapon SG, Clariant, (INCI: Sodium Cocoyl | | 5.00% |
| Glycinate) | | |
| Microcare ZP, Thor, (INCI: Zinc Pyrithione) | | 0.20% |
| Octopirox, Clariant, (INCI: Octopirox) | | 0.10% |
| ABIL ® Quat 3272, Evonik Nutrition & | | 0.80% |
| Care GmbH, (INCI: Quaternium-80) | | |
| REWOMID ® D 212, Evonik Nutrition & | | 0.80% |
| Care GmbH, (INCI: Cocamide MEA) | | |
| ANTIL ® 500, Evonik Nutrition & Care GmbH, | | 0.80% |
| (INCI: PEG-200 Glyceryl Stearate) | | |
| Glycerol | | 1.50% |
| NaCl | | 0.90% |
| Citric acid, 30% strength | | q.s. to |
| | | pH 5.5 |
| Preservative | | q. s. |

### Formulation Example 18) PEG-Free Shampoo

| | | |
|---|---|---|
| TEXAPON ® LS 35, BASF, 30% strength | | 12.00% |
| (INCI: Sodium Lauryl Sulfate) | | |
| TEGO ® Betain F 50, Evonik Nutrition & Care GmbH, | | 18.00% |
| 38% strength (INCI: Cocamidopropyl Betaine) | | |
| Iselux, Innospec, (INCI: Sodium Lauroyl Methyl | | 4.00% |
| Isethionate) | | |
| TEGOSOFT ® PC 41, Evonik Nutrition & Care | | 2.00% |
| GmbH, (INCI: Polyglyceryl-4 Caprate) | | |
| Microemulsion ME18 | | 1.50% |
| Perfume | | 0.25% |
| Water | to | 100.00% |
| Jaguar C-162, Solvay (INCI: Hydroxypropyl | | 0.20% |
| Guar Hydroxypropyltrimonium Chloride) | | |
| Versene 100, The Dow Chemical Company, | | 0.20% |
| (INCI: Tetrasodium EDTA) | | |
| REWOMID ® D 212, Evonik Nutrition & Care GmbH, | | 0.80% |
| (INCI: Cocamide MEA) | | |
| ANTIL ® SPA 80, Evonik Nutrition & Care GmbH, | | 0.80% |
| (INCI: Isostearamide MIPA; Glyceryl Laurate) | | |
| Keltrol CG-SFT, CP Kelco, (INCI: Xanthan Gum) | | 0.20% |
| Glycerol | | 1.50% |
| NaCl | | 0.70% |
| Citric acid, 30% strength | | q.s. to |
| | | pH 5.1 |
| Preservative | | q. s. |

### Formulation Example 19) Conditioning Anti-Dandruff Shampoo

| | | |
|---|---|---|
| A | TEGIN ® G 1100 Pellets, Evonik Nutrition & Care | 3.00% |
| | GmbH, | |
| | (INCI: Glycol Distearate) | |
| | TEXAPON ® NSO, BASF, 28% strength, | 40.00% |
| | (INCI: Sodium Laureth Sulfate) | |
| B | Perfume | 0.30% |
| | Zinc-Pyrion NF, WeyIChem, 48% strength, (INCI: | 2.00% |
| | Zinc Pyrithione) | |
| | Microemulsion ME3 | 2.00% |
| C | Water | to 100.00% |
| | TEGO ® Carbomer 341 ER, Evonik Nutrition & Care | 0.20% |
| | GmbH, | |
| | (INCI: Acrylates/C10-30 Alkyl Acrylate | |
| | Crosspolymer) | |
| | Water | 0.30% |
| | NaOH, 25% strength | 0.30% |
| D | REWOTERIC ® AM B U 185, Evonik Nutrition & | 12.50% |
| | Care GmbH, 30% strength, (INCI: | |
| | Undecylenamidopropyl Betaine) | |
| | ANTIL ® SPA 80, Evonik Nutrition & Care GmbH, | 3.70% |
| | (INCI: Isostearamide MIPA; Glyceryl Laurate) | |
| | Preservative | q. s. |

### Formulation Example 20) PEG- and Sulfate-Free Shampoo

| | |
|---|---|
| REWOTERIC ® AM C, Evonik | 16.00% |
| Nutrition & Care GmbH, | |
| 32% strength, (INCI: Sodium Cocoamphoacetate | |
| Plantapon ACG 50, BASF, | 3.80% |
| (INCI: Disodium Cocoyl Glutamate) | |
| Plantacare ® 818 UP, BASF, 51% strength, | 5.00% |
| (INCI: Coco Glucoside) | |
| Microemulsion ME2 | 2.50% |
| Perfume | 0.30% |
| Water | to 100.00% |
| TEGO ® Betain F 50, Evonik Nutrition & Care | 6.00% |
| GmbH, 38% strength, | |
| (INCI: Cocamidopropyl Betaine) | |
| ANTIL ® SPA 80, Evonik Nutrition & Care GmbH, | 1.80% |
| (INCI: Isostearamide MIPA; Glyceryl Laurate) | |
| Preservative | q. s. |
| Citric acid, 30% strength | q. s. to |
| | pH 5.0 |

### Formulation Example 21) PEG- and Sulfate-Free Shampoo

| | | |
|---|---|---|
| A | REWOTERIC ® AMC, Evonik Nutrition & Care | 20.00% |
| | GmbH, 32% strength, (INCI: Sodium | |
| | Cocoamphoacetate) | |
| | REWOPOL ® SB F 12 P, Evonik Nutrition & Care | 6.00% |
| | GmbH, 96% strength, (INCI: Disodium Lauryl | |
| | Sulfosuccinate) | |
| | Microemulsion ME18 | 2.00% |
| | ANTIL ® SPA 80, Evonik Nutrition & Care GmbH, | 1.70% |
| | (INCI: Isostearamide MIPA; Glyceryl Laurate) | |
| B | Water | to 100.00% |
| | Jaguar C-162, Solvay, (INCI: Hydroxypropyl Guar | 0.10% |
| | Hydroxypropyltrimonium Chloride) | |
| | Citric acid, 30% strength | 3.60% |
| C | ANTIL ® HS 60, Evonik Nutrition & Care GmbH, | 3.00% |
| | (INCI: Cocamidopropyl Betaine; Glyceryl Laurate) | |
| | Preservative | q. s. |

### Formulation Example 22) Mild Hair & Body Wash, PEG- and Sulfate-Free

| | |
|---|---|
| Plantacare ® 1200 UP, BASF, 50% strength, (INCI: Lauryl | 11.40% |
| Glucoside) | |
| Plantacare ® 818 UP, BASF, 51% strength, (INCI: Coco | 5.60% |
| Glucoside) | |
| Water | to 100.00% |
| ANTIL ® Soft SC, Evonik Nutrition & Care GmbH, | 0.90% |
| (INCI: Sorbitan Sesquicaprylate) | |
| Microemulsion ME3 | 1.00% |
| TEGOSOFT ® LSE 65 K SOFT, Evonik Nutrition & Care | 1.50% |
| GmbH, (INCI: Sucrose Cocoate) | |
| TEGO ® Betain F 50, Evonik Nutrition & Care GmbH, 38% | 18.00% |
| strength, (INCI: Cocamidopropyl Betaine) | |
| Perfume, preservative | q. s. |
| Citric acid, 30% strength | q.s. to |
| | pH 5.0 |

### Formulation Example 23) Sulfate-Free Mild Shampoo

| | |
|---|---|
| Plantacare ® 1200 UP, BASF, 50% strength, | 8.50% |
| (INCI: Lauryl Glucoside) | |
| Plantacare ® 818 UP, BASF, 51% strength, | 2.30% |
| (INCI: Coco Glucoside) | |
| Water | to 100.00% |
| ANTIL ® 200, Evonik Nutrition & Care GmbH, | 4.00% |
| (INCI: PEG-200 Hydrogenated | |
| Glyceryl Palmate; PEG-7 Glyceryl Cocoate) | |
| Microemulsion ME3 | 5.00% |
| REWOPOL ® SB FA 30 B, Evonik Nutrition & Care | 9.00% |
| GmbH, (INCI: Disodium Laureth Sulfosuccinate) | |
| TEGO ® Betain F 50, | 14.70% |
| Evonik Nutrition & Care GmbH, 38% | |
| strength, (INCI: Cocamidopropyl Betaine) | |
| Perfume, preservative | q. s. |
| Citric acid, 30% strength | q.s. to pH 5.0 |

### Formulation Example 24) Sulfate-Free Mild Shampoo

| | |
|---|---|
| Plantacare ® 1200 UP, BASF, 50% strength, (INCI: Lauryl | 8.50% |
| Glucoside) | |
| Plantacare ® 818 UP, BASF, 51% strength, (INCI: Coco | 2.30% |
| Glucoside) | |
| Water | to 100.00% |
| Tilamar 740, DSM, | 0.20% |
| (INCI: Polyquatemium-7) | |
| ANTIL ® 171, Evonik Nutrition & Care GmbH, | 0.60% |
| (INCI: PEG-18 Glyceryl Oleate/Cocoate) | |
| Microemulsion ME3 | 3.00% |
| Perlastan SC 25 NKW, Schill&Seilacher, | 14.40% |
| (INCI: Disodium Cocoyl Glutamate, Sodium Cocoyl | |
| Glutamate) | |
| TEGO ® Betain F 50, Evonik Nutrition & Care GmbH, 38% | 14.70% |
| strength, (INCI: Cocamidopropyl Betaine) | |
| Perfume, preservative | q.s. |
| Citric acid, 30% strength | q.s. to pH 5.3 |

### Formulation Example 25) PEG- and Sulfate-Free Mild Shampoo

| | |
|---|---|
| Plantacare ® 1200 UP, BASF, 50% strength, (INCI: Lauryl | 10.00% |
| Glucoside) | |
| Water | to 100.00% |
| UCARE Polymer JR-400, Dow Chemicals, | 0.10% |
| (INCI: Polyquatemium-10) | |
| ANTIL ® SPA 80, Evonik Nutrition & Care GmbH, | 1.20% |
| (INCI: Isostearamide MIPA; Glyceryl Laurate) | |
| Microemulsion ME2 | 2.50% |
| REWOTERIC ® AM C, Evonik Nutrition & Care GmbH, | 21.30% |
| 32% strength, (INCI: Sodium Cocoamphoacetate | |
| TEGO ® Betain P 50 C, Evonik Nutrition & Care GmbH, | 10.50% |
| 38% strength, (INCI: Cocamidopropyl Betaine) | |
| Perfume, preservative | q.s. |
| Citric acid, 30% strength | q.s. to pH 5.1 |

### Formulation Example 26) Shampoo

| | |
|---|---|
| TEXAPON ® NSO, BASF, 28% strength, | 32.10% |
| (INCI: Sodium Laureth Sulfate) | |
| Water | to 100.00% |
| UCARE Polymer JR-400, Dow Chemicals, | 0.20% |
| (INCI: Polyquaternium-10) | |
| ANTIL ® SPA 80, Evonik Nutrition & Care GmbH, | 0.50% |
| (INCI: Isostearamide MIPA; Glyceryl Laurate) | |
| ANTIL ® 171, Evonik Nutrition & Care GmbH, | 2.00% |
| (INCI: PEG-18 Glyceryl Oleate/Cocoate) | |
| Microemulsion ME3 | 5.00% |
| TEGO ® Pearl N 300, Evonik Nutrition & Care GmbH, | 1.50% |
| (INCI: Glycol Distearate; Laureth-4; Cocamidopropyl | |
| Betaine) | |
| TEGO ® Betain F 50, Evonik Nutrition & Care GmbH, | 7.90% |
| 38% strength, (INCI: Cocamidopropyl Betaine) | |
| Sodium Chloride | 1.00% |
| Perfume, preservative | q.s. |
| Citric acid, 30% strength | q.s. to pH 5.0 |

### Formulation Example 27) PEG-Free Shampoo

| | |
|---|---|
| TEXAPON ® LS 35, BASF, 30%, | 28.00% |
| (INCI: Sodium Lauryl Sulfate) | |
| Water | to 100.00% |
| UCARE Polymer JR-400, Dow Chemicals, | 0.10% |
| (INCI: Polyquaternium-10) | |
| Jaguar C-162, Solvay, | 0.10% |
| (INCI: Hydroxypropyl Guar Hydroxypropyltrimonium | |
| Chloride) | |
| ANTIL ® CM 90, Evonik Nutrition & Care GmbH, | 0.50% |
| (INCI: Cocamide MEA) | |
| ANTIL ® SPA 80, Evonik Nutrition & Care GmbH, | 1.00% |
| (INCI: Isostearamide MIPA; Glyceryl Laurate) | |
| Xanthan Gum | 0.50% |
| Microemulsion ME3 | 3.00% |
| Argan Oil, DSM Nutritional Products Ltd., (INCI: Argania | 0.10% |
| Spinosa Oil) | |
| Dehyton AB 30, BASF, 31%, (INCI: Coco-Betaine) | 8.00% |
| Prifrac 2920, Croda, (INCI: Lauric Acid) | 0.50% |
| TEGOSOFT ® PC 41, Evonik Nutrition & Care GmbH, | 1.00% |
| (INCI: Polyglyceryl-4 Caprate) | |
| Glycerol | 1.00% |
| Uvinul MS 40, BASF, (INCI: Benzophenon-4) | 0.10% |
| Versene 100, The Dow Chemical Company, (INCI: | 0.1% |
| Tetrasodium EDTA) | |
| Sodium Chloride | 1.00% |
| Perfume, preservative | q. s. |
| Citric acid, 30% strength | q.s. to pH 5.0 |

### Formulation Example 28) Sulfate-Free Shampoo

| | |
|---|---|
| Bioterge AS-40 AOS, Stepan, | 30.00% |
| (INCI: Sodium C14-16 Olefin Sulfonate) | |
| Water | to 100.00% |
| Jaguar C-162, Solvay, | 0.15% |
| (INCI: Hydroxypropyl Guar Hydroxypropyltrimonium | |
| Chloride) | |
| ANTIL ® 500 Pellets, Evonik Nutrition & Care GmbH, | 0.20% |
| (INCI: PEG-200 Glyceryl Stearate) | |
| ANTIL ® SPA 80, Evonik Nutrition & Care GmbH, | 0.50% |
| (INCI: Isostearamide MIPA; Glyceryl Laurate) | |
| Microemulsion ME2 | 3.50% |
| Cropure Avocado, Croda, (INCI: Persea Gratissima | 0.10% |
| (Avocado) Oil) | |
| Dehyton AB 30, BASF, 31%, (INCI: Coco-Betaine) | 8.00% |
| TAGAT ® CH 40, Evonik Nutrition & Care GmbH, | 1.00% |
| (INCI: PEG-40 Hydrogenated Castor Oil) | |
| Glycerol | 1.00% |
| Uvinul MS 40, BASF, (INCI: Benzophenon-4) | 0.10% |
| Sodium Chloride | 1.50% |
| Perfume, preservative | q. s. |
| Citric acid, 30% strength | q.s. to pH 5.5 |

### Formulation Example 29) Sulfate-Free Shampoo

| | |
|---|---|
| TEGO ® Betain F 50, Evonik Nutrition & Care GmbH, | 22.00% |
| 38% strength, (INCI: Cocamidopropyl Betaine) | |
| Water | to 100.00% |
| Jaguar C-162, Solvay, | 0.10% |
| (INCI: Hydroxypropyl Guar Hydroxypropyltrimonium | |
| Chloride) | |
| ANTIL ® 500 Pellets, Evonik Nutrition & Care GmbH, | 0.30% |
| (INCI: PEG-200 Glyceryl Stearate) | |
| REWOPAL ® PEG 6000 DS A, Evonik Nutrition & Care | 0.50% |
| GmbH, | |
| (INCI: PEG-150 Distearate) | |
| Microemulsion ME3 | 2.50% |
| Cropure Olive, Croda, (INCI: Olea Europaea (Olive) Fruit | 0.10% |
| Oil) | |
| Hostapon SG, Clariant, (INCI: Sodium Cocoyl Glycinate) | 10.00% |
| TEGO ® Solve 61, Evonik Nutrition & Care GmbH, | 1.00% |
| (INCI: Polyglycery1-6 Caprylate; Polyglycery1-3 Cocoate; | |
| Polyglycery1-4 Caprate; Polyglycery1-6 Ricinoleate) | |
| Glycerol | 0.50% |
| Sodium Chloride | 1.00% |
| Perfume, preservative | q. s. |
| Citric acid, 30% strength | q.s. to pH 4.8 |

### Formulation Example 30) Rinse-Off Conditioner

| | |
|---|---|
| Water | to 100.00% |
| VARISOFT ® BT 85, Evonik Nutrition & Care GmbH, | 2.00% |
| (INCI: Behentrimonium Chloride) | |
| Microemulsion ME2 | 3.50% |
| TEGO ® Alkanol 1618, Evonik Nutrition & Care GmbH, | 5.00% |
| (INCI: Cetearyl Alcohol) | |
| Citric acid, 30% strength | q.s. to pH 4.5 |
| Preservative, perfume | q.s. |

### Formulation Example 31) Rinse-Off Conditioner

| | |
|---|---|
| Water | to 100.00% |
| VARISOFT ® EQ 65, Evonik Nutrition & Care GmbH, | 2.00% |
| (INCI: Distearoylethyl Dimonium Chloride; Cetearyl | |
| Alcohol) | |
| VARISOFT ® BT 85, Evonik Nutrition & Care GmbH, | 1.00% |
| (INCI: Behentrimonium Chloride) | |
| Microemulsion ME19 | 1.80% |
| TEGO ® Alkanol 1618, Evonik Nutrition & Care GmbH, | 5.00% |
| (INCI: Cetearyl Alcohol) | |
| Citric acid, 30% strength | q.s. to pH 4.0 |
| Preservative, perfume | q.s. |

### Formulation Example 32) Rinse-Off Conditioner

| | |
|---|---|
| Water | to 100.00% |
| VARISOFT ® EQ 65, Evonik Nutrition & Care GmbH, | 2.00% |
| (INCI: Distearoylethyl Dimonium Chloride; Cetearyl | |
| Alcohol) | |
| VARISOFT ® BT 85, Evonik Nutrition & Care GmbH | 2.00% |
| (INCI: Behentrimonium Chloride) | |
| ABIL ® Quat 3272, Evonik Nutrition & Care GmbH, | 0.50% |
| (INCI: Quaternium-80) | |
| Microemulsion ME18 | 4.50% |
| TEGINACID ® C, Evonik Nutrition & Care GmbH, | 0.50% |
| (INCI: Ceteareth-25) | |
| TEGO ® Alkanol 1618, Evonik Nutrition & Care GmbH, | 5.00% |
| (INCI: Cetearyl Alcohol) | |
| Citric acid, 30% strength | q.s. to pH 3.5 |
| Preservative, perfume | q.s. |

### Formulation Example 33) Rinse-Off Conditioner

| | |
|---|---|
| TEGINACID ® C, Evonik Nutrition & Care GmbH, | 0.50% |
| (INCI: Ceteareth-25) | |
| TEGO ® Alkanol 16, Evonik Nutrition & Care GmbH, | 2.00% |
| (INCI: Cetyl Alcohol) | |
| TEGO ® Amid S 18, Evonik Nutrition & Care GmbH, | 1.00% |
| (INCI: Stearamidopropyl Dimethylamine) | |
| Microemulsion ME2 | 3.50% |
| Propylene Glycol | 2.00% |
| Citric Acid Monohydrate | 0.30% |
| Water | to 100.00% |
| Preservative, perfume | q.s. |

### Formulation Example 34) Rinse-Off Conditioner

| | |
|---|---|
| TEGINACID ® C, Evonik | 0.50% |
| Nutrition & Care GmbH, (INCI: | |
| Ceteareth-25) | |
| TEGO ® Alkanol 1618, Evonik | 6.50% |
| Nutrition & Care GmbH, (INCI: | |
| Cetearyl Alcohol) | |
| Microemulsion ME19 | 3.50% |
| Water | to 100.00% |
| TEGO ® Cosmo C 100, Evonik | 0.50% |
| Nutrition & Care GmbH, (INCI: | |
| Creatine) | |
| Propylene Glycol | 2.00% |
| Citric acid, 30% strength | q.s. to pH 4.0 |
| Preservative, perfume | q.s. |

### Formulation Example 35) Rinse-Off Conditioner

| | |
|---|---|
| Water | to 100.00% |
| TEGIN ® M Pellets, Evonik Nutrition & | 1.00% |
| Care GmbH, (INCI: Glyceryl Stearate) | |
| Oleyl Alcohol | 0.70% |
| Glycerol | 1.50% |
| Microemulsion ME2 | 2.50% |
| TEGO ® Alkanol 1618, Evonik | 4.50% |
| Nutrition & Care GmbH, (INCI: | |
| Cetearyl Alcohol) | |
| Citric acid, 30% strength | q.s. to pH 4.0 |
| Preservative, perfume | q.s. |

### Formulation Example 36) Rinse-Off Conditioner

| | |
|---|---|
| Water | to 100.00% |
| TEGIN ® M Pellets, Evonik | 1.00% |
| Nutrition & Care GmbH, (INCI: | |
| Glyceryl Stearate) | |
| Oleyl Alcohol | 0.50% |
| Glycerol | 2.00% |
| Pure-Gel, Grain Processing | 1.50% |
| Corporation, (INCI: Sodium | |
| Hydroxypropyl Starch Phosphate) | |
| Microemulsion ME2 | 2.80% |
| TEGO ® Alkanol 1618, Evonik | 6.00% |
| Nutrition & Care GmbH, (INCI: | |
| Cetearyl Alcohol) | |
| Citric acid, 30% strength | q.s. to pH 4.0 |
| Preservative, perfume | q.s. |

### Formulation Example 37) Rinse-Off Conditioner

| | |
|---|---|
| Water | to 100.00% |
| TEGINACID ® C, Evonik | 1.00% |
| Nutrition & Care GmbH, (INCI: | |
| Ceteareth-25) | |
| SF 1708, Momentive, | 1.00% |
| (INCI: Amodimethicone) | |
| DC 1503 Fluid, Dow Corning, (INCI: | 0.50% |
| Dimethicone, Dimethiconol) | |
| Microemulsion ME3 | 3.80% |
| TEGO ® Alkanol 1618, Evonik | 6.00% |
| Nutrition & Care GmbH, (INCI: | |
| Cetearyl Alcohol) | |
| Citric acid, 30% strength | q.s. to pH 4.0 |
| Preservative, perfume | q.s. |

### Formulation Example 38) Rinse-Off Conditioner

| | |
|---|---|
| Water | to 100.00% |
| TEGIN ® M Pellets, Evonik Nutrition & | 1.00% |
| Care GmbH, (INCI: Glyceryl Stearate) | |
| TEGO ® Care PSC 3, Evonik Nutrition & | 0.50% |
| Care GmbH, (INCI: Polyglyceryl-3 | |
| Dicitrate/Stearate) | |
| Jaguar C-162, Solvay, | 0.10% |
| (INCI: Hydroxypropyl Guar | |
| Hydroxypropyltrimonium Chloride) | |
| Microemulsion ME2 | 5.00% |
| TEGO ® Alkanol 1618, Evonik | 6.00% |
| Nutrition & Care GmbH, (INCI: | |
| Cetearyl Alcohol) | |
| Citric acid, 30% strength | q.s. to pH 4.5 |
| Preservative, perfume | q.s. |

### Formulation Example 39) Rinse-Off Conditioner

| | |
|---|---|
| Water | to 100.00% |
| TEGIN ® M Pellets, Evonik Nutrition & | 1.00% |
| Care GmbH, (INCI: Glyceryl Stearate) | |
| Oleyl Alcohol | 0.30% |
| Lauryl Alcohol | 0.30% |
| TEGO ® Care PSC 3, Evonik Nutrition & | 0.80% |
| Care GmbH, (INCI: Polyglyceryl-3 | |
| Dicitrate/Stearate) | |
| Glycerol | 0.50% |
| Microemulsion ME2 | 5.50% |
| TEGO ® Alkanol 1618, Evonik | 6.00% |
| Nutrition & Care GmbH, (INCI: | |
| Cetearyl Alcohol) | |
| Citric acid, 30% strength | q.s. to pH 4.5 |
| Preservative, perfume | q.s. |

### Formulation Example 40) Rinse-Off Conditioner

| | |
|---|---|
| Water | to 100.00% |
| CORN PO4 PH “B”, Agrana Starch, | 4.00% |
| (INCI: Distarch Phosphate) | |
| Microemulsion ME2 | 2.50% |
| TEGO ® Alkanol 16, Evonik | 1.00% |
| Nutrition & Care GmbH,(INCI: | |
| Cetyl Alcohol) | |
| TEGO ® Alkanol 18, Evonik | 1.70% |
| Nutrition & Care GmbH, (INCI: | |
| Stearyl Alcohol) | |
| Citric acid, 30% strength | q.s. to pH 4.3 |
| Preservative, perfume | q.s. |

### Formulation Example 41) Leave-In Conditioner Spray

| | |
|---|---|
| Lactic Acid, 80% | 0.40% |
| Water | to 100.00% |
| TEGO ® Amid S 18, Evonik Nutrition & | 1.00% |
| Care GmbH, (INCI: Stearamidopropyl | |
| Dimethylamine) | |
| TEGIN ® G 1100 Pellets, Evonik Nutrition & | 0.60% |
| Care GmbH, (INCI: Glycol Distearate) | |
| TEGO ® Care PS, Evonik Nutrition & | 1.20% |
| Care GmbH, (INCI: Methyl Glucose | |
| Sesquistearate) | |
| Glycerol | 1.50% |
| Microemulsion ME2 | 3.00% |
| Preservative, perfume | q.s. |

### Formulation Example 42) Leave-In Conditioner Spray

| | |
|---|---|
| TAGAT ® CH-40, Evonik Nutrition & Care | 2.00% |
| GmbH, (INCI: PEG-40 Hydrogenated Castor | |
| Oil) | |
| Ceramide VI, Evonik Nutrition & Care | 0.10% |
| GmbH, (INCI: Ceramide 6 II) | |
| Perfume | 0.20% |
| Glycerol | 1.00% |
| Water | to 100.00% |
| Microemulsion ME2 | 5.00% |
| LACTIL ®, Evonik Nutrition & Care GmbH, | 2.00% |
| (INCI: Sodium Lactate; Sodium PCA; Glycine; | |
| Fructose;Urea; Niacinamide; Inositol; Sodium | |
| benzoate; Lactic Acid) | |
| TEGO ® Betain F 50, Evonik Nutrition & | 2.30% |
| Care GmbH, 38% (INCI: Cocamidopropyl | |
| Betaine) | |
| Citric acid, 10% strength | 2.00% |

### Formulation Example 43) Leave-In Conditioner Foam

| | |
|---|---|
| Microemulsion ME3 | 3.50% |
| TEGOSOFT ® PC 41, Evonik Nutrition & | 3.00% |
| Care GmbH, (INCI: Polyglyceryl-4 Caprate) | |
| Perfume | 0.20% |
| TEGO ® Betain 810, Evonik Nutrition & | 2.00% |
| Care GmbH, (INCI: Capryl/Capramidopropyl | |
| Betaine) | |
| Water | to 100.00% |
| TEGO ® Cosmo C 100, Evonik Nutrition & | 0.50% |
| Care GmbH, (INCI: Creatine) | |
| TEGOCEL ® HPM 50, Evonik Nutrition & | 0.30% |
| Care GmbH,(INCI: Hydroxypropyl | |
| Methylcellulose) | |
| Citric acid, 30% strength | 0.10% |
| Preservative | q. s. |

### Formulation Example 44) Strong Hold Styling Gel

| | |
|---|---|
| TEGO ® Carbomer 141, Evonik Nutrition & | 1.20% |
| Care GmbH, (INCI: Carbomer) | |
| Water | to 100.00% |
| NaOH, 25% strength | 2.60% |
| PVP/VA W-735, ISP, | 16.00% |
| (INCI: PVP/VA Copolymer) | |
| Microemulsion ME2 | 4.80% |
| Alcohol Denat. | 10.00% |
| TAGAT ® O 2 V, Evonik Nutrition & Care | 2.00% |
| GmbH, (INCI: PEG-20 Glyceryl Oleate) | |
| Perfume | 0.30% |
| ABIL ® B 88183 PH, Evonik Nutrition & | 0.30% |
| Care GmbH, (INCI: PEG/PPG-20/6 | |
| Dimethicone) | |
| Preservative | q. s. |

### Formulation Example 45) Sprayable Hairmilk, PEG-Free

| | | |
|---|---|---|
| A | Water | to 100.00% |
| | Lactic acid, 80% strength | 0.40% |
| B | Glycerol | 1.20% |
| | TEGIN ® G 1100 Pellets, Evonik | 0.50% |
| | Nutrition & Care GmbH, (INCI: Glycol | |
| | Distearate) | |
| | TEGO ® Care PS, Evonik Nutrition & | 1.20% |
| | Care GmbH, (INCI: Methyl Glucose | |
| | Sesquistearate) | |
| | Microemulsion ME18 | 2.50% |
| | Perfume, preservative | q. s. |

### Formulation Example 46) Hair Colorant

| Water | to 100.00% |
|---|---|
| TEGO ® Alkanol 1618, Evonik Nutrition & | 10.00% |
| Care GmbH, (INCI: Cetearyl Alcohol) | |
| Eutanol ® G, BASF, (INCI: Octyldodecanol) | 3.50% |
| REWOMID ® C 212, Evonik Nutrition & | 1.70% |
| Care GmbH, (INCI: Cocamide MEA) | |
| Super Hartolan ® B, Croda, (INCI: Lanolin | 3.00% |
| Alcohol) | |
| Avocado oil, Henry Lamotte, (INCI: Persea | 1.50% |
| Gratissima Oil) | |
| Pristerene ® 4960, Uniquema, (INCI: Stearic | 6.00% |
| Acid) | |
| EDTA BD, BASF, (INCI: Disodium EDTA) | 0.10% |
| Texapon ® K12G, BASF, (INCI: Sodium | 0.60% |
| Lauryl sulfate) | |
| Propylene glycol | 5.00% |
| Timica Silver Sparkle, BASF, (INCI: MICA; | 1.00% |
| TitaniumDioxide) | |
| Ammonia solution, 25% strength | 6.00% |
| 2,5-Diaminotoluene sulfate, (INCI: Toluene- | 1.40% |
| 2,5-Diamine) | |
| Rodol ® RS, Jos. H. Lowenstein & Sons, | 0.30% |
| (INCI: Resorcinol) | |
| HC Blue A42, (INCI: 2,4- | 0.10% |
| Diaminophenoxyethanol di HCl) | |
| Sodium sulfite | 0.50% |
| Perfume | 0.20% |
| Microemulsion ME3 | 1.00% |

### Formulation Example 47) Hair Colorant

| Water | to 100.00% |
|---|---|
| TEGO ® Alkanol 1618, Evonik | 12.00% |
| Nutrition & Care GmbH, (INCI: Cetearyl | |
| Alcohol) | |
| Super Hartolan ® B, Croda, (INCI: Lanolin | 2.50% |
| Alcohol) | |
| Meadowfoam ® Seed Oil, Fanning, (INCI: | 1.00% |
| Limnanthes Alba) | |
| Pristerene ® 4960, Uniquema, (INCI: | 5.50% |
| Stearic Acid) | |
| EDTA BD, BASF, (INCI: Disodium | 0.30% |
| EDTA) | |
| Glycerol | 3.00% |
| Texapon ® N 70, BASF, (INCI: Sodium | 2.00% |
| Laureth Sulfate) | |
| Monoethanolamine | 4.00% |
| 2,5-Diaminotoluene sulfate, (INCI: | 0.90% |
| Toluene-2,5-Diamine) | |
| Rodol ® RS, Jos. H. Lowenstein & Sons, | 0.20% |
| (INCI: Resorcinol) | |
| Jarocol ® 4A3MP, Vivimed Labs, | 0.60% |
| (INCI: 4-Amino-M-Cresol) | |
| Rodol ® PAOC, Jos. H. Lowenstein & | 0.50% |
| Sons, (INCI: 4-Amino-2-Hydroxytoluene) | |
| Uantox ® EBATE, Universal Preserv- | 0.50% |
| A-Chem, (INCI: Erythorbic Acid) | |
| Perfume | 0.20% |
| Microemulsion ME2 | 1.00% |

### Formulation Example 48) Hair Colorant

| Water | to 100.00% |
|---|---|
| TEGO ® Alkanol 1618, Evonik | 10.00% |
| Nutrition & Care GmbH, (INCI: Cetearyl | |
| Alcohol) | |
| Eutanol ® G, BASF, (INCI: | 1.00% |
| Octyldodecanol) | |
| REWOMID ® C 212, Evonik Nutrition & | 2.00% |
| Care GmbH, (INCI: Cocamide MEA) | |
| TEGIN ® VS, Evonik Nutrition & Care | 5.00% |
| GmbH, (INCI: Glyceryl Stearate SE) | |
| Fitoderm ®, Hispano Quimica S. A., | 1.00% |
| (INCI: Squalane) | |
| Coenzyme Q 10 | 0.10% |
| EDTA BD, BASF, (INCI: Disodium | 0.30% |
| EDTA) | |
| Texapon ® K12G, BASF, (INCI: Sodium | 0.20% |
| Lauryl Sulfate) | |
| Propylene glycol | 5.00% |
| Ammonia solution, 25% strength | 3.00% |
| Rodol ® ERN, Jos. H. Lowenstein & | 0.30% |
| Sons, (INCI: 1-Naphthol) | |
| Imexine ® OAG, Chimex, | 1.00% |
| (INCI: 2-Methyl-5- | |
| Hydroxyethylaminophenol) | |
| Colorex ® WP5, Teluca, | 2.60% |
| (INCI: 1-Hydroxyethyl 4,5-Diamino | |
| Pyrazole Sulfate) | |
| Ascorbic acid | 0.20% |
| Perfume | 0.30% |
| Microemulsion ME6 | 2.50% |

1. A composition comprising
A) at least one non-silicone-containing quaternary ammonium compound,
B) at least one cosmetic oil,
C) at least one nonionic surfactant,
D) at least one nonaqueous solvent, and
E) water.

2. The composition according to claim 1, wherein the composition is a microemulsion.

3. The composition according to claim 1, wherein the composition contains
A) in an amount of from 1 wt % to 40 wt %,
B) in an amount of from 5 wt % to 60 wt %,
C) in an amount of from 5 wt % to 50 wt %,
D) in an amount of from 2 wt % to 50 wt %, and
E) in an amount of from 1 wt % to 60 wt %,
wherein the percentages by weight refer to the total composition.

4. The composition according to claim 1, wherein
A) is selected from the group of the ester quats and imidazolinium salts,

5. The composition according to claim 1, wherein
the mass of the component B) is greater than the mass of the component A) in the composition.

6. The composition according to claim 1, wherein
A) is selected from the group of the liquid ester quats consisting of quaternized fatty acid alkanolamine ester salts.

7. The composition according to claim 1, wherein
A) is selected from the group of the liquid ester quats comprising compounds of the general formula (I) 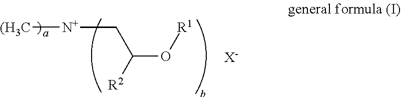
wherein R¹ is an acyl radical of an at least monounsaturated fatty acid having a chain length of 18 to 24 carbon atoms or the acyl radical of isostearic acid or ricinoleic acid,
wherein R² is an alkyl radical having 1 to 6 carbon atoms, and
wherein X⁻ is a counterion to the positive charge on the quaternary nitrogen group,
wherein a=1 to 3 and b=1 to 3,
with the proviso that a+b=4.

8. The composition according to claim 1, wherein
component C) is selected from those of the general formula IV 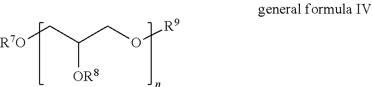
wherein n=2 to 16, and
R⁷, R⁸, R⁹=are independently the same or different and are selected from H and saturated or unsaturated, linear or branched acyl radical having 4-36 carbon atoms and containing at least one hydroxyl group.

9. The composition according to claim 1, wherein
D) is selected from the group consisting of hydrotropes and polyols.

10. The composition according to claim 1, wherein the composition comprises
F) amphoteric surfactant,

11. The process for producing care and cleaning formulations, comprising the process steps of:
1) providing a composition according to claim 1,
2) mixing the composition with an aqueous phase.

12. A care and cleaning formulations containing a composition according to claim 1.

13. A care and cleaning formulations containing a composition obtained by the process according to claim 11.

14. The composition according to claim 1, wherein the composition contains
A) in an amount of from 1.5 wt % to 30 wt %,
B) in an amount of from 10 wt % to 50 wt %,
C) in an amount of from 10 wt % to 40 wt %,
D) in an amount of from 3 wt % to 40 wt %, and
E) in an amount of from 3 wt % to 50 wt %,
wherein the percentages by weight refer to the total composition.

15. The composition according to claim 1, wherein the composition contains
A) in an amount of from 5 wt % to 25 wt %,
B) in an amount of from 15 wt % to 40 wt %,
C) in an amount of from 15 wt % to 35 wt %,
D) in an amount of from 5 wt % to 30 wt %, and
E) in an amount of from 5 wt % to 45 wt %,
wherein the percentages by weight refer to the total composition.

16. The composition according to claim 1, wherein
A) is selected from the group of liquid ester quats and liquid imidazolinium salts.

17. The composition according to claim 1, wherein
A) is selected from the group of the quaternized fatty acid ethanolamine ester salts and the quaternized fatty acid isopropanolamine ester salts.

18. The composition according to claim 1, wherein
A) is selected from the group of the liquid ester quats comprising compounds of the general formula (I) 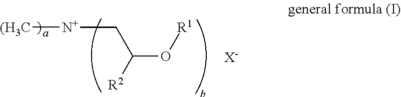
where R¹ is an acyl radical of an at least monounsaturated fatty acid having a chain length of 18 to 24 carbon atoms or the acyl radical of isostearic acid or ricinoleic acid,
wherein R² is methyl, ethyl, propyl or isopropyl, and
wherein X⁻ is a counterion to the positive charge on the quaternary nitrogen group,
wherein a=1.7 to 2.3 and b=1.7 to 2.3,
with the proviso that a+b=4.

19. The composition according to claim 1, wherein
A) is selected from the group of the liquid ester quats comprising compounds of the general formula (I) 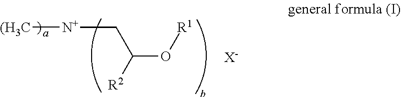
wherein R¹ is an acyl radical of an at least monounsaturated fatty acid having a chain length of 18 to 24 carbon atoms or the acyl radical of isostearic acid or ricinoleic acid,
wherein R² is methyl, and
wherein X⁻ is a counterion to the positive charge on the quaternary nitrogen group,
wherein a=1.7 to 2.3 and b=1.7 to 2.3,
with the proviso that a+b=4.

20. The composition according to claim 1, wherein
component C) is selected from those of the general formula IV 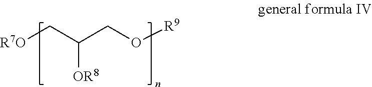
where
n=3 to 14, and
R⁷, R⁸, R⁹=are selected from the acyl radicals of natural fatty acids.
